(19) United States
(12) Patent Application Publication
(10) Pub. No.: US 2022/0136069 A1
(43) Pub. Date: May 5, 2022

(54) MACROPHAGE EXPRESSION IN BREAST CANCER

(71) Applicant: The University Court of the University of Edinburgh, Edinburgh (GB)

(72) Inventors: Jeff Pollard, Edinburgh (GB); Luca Cassetta, Edinburgh (GB); Stamatina Fragkogianni, Edinburgh (GB)

(21) Appl. No.: 17/436,161

(22) PCT Filed: Mar. 6, 2020
(86) PCT No.: PCT/EP2020/056125

§ 371 Date: Sep. 3, 2021

(30) Foreign Application Priority Data
Mar. 6, 2019 (GB) 1903015.4

(51) Int. Cl. C12Q 1/6886

(52) U.S. Cl. CPC C12Q 1/6886; C12Q 2600/158; C12Q 2600/118

(57) ABSTRACT

The present invention relates to the field of breast cancer diagnosis and treatment. The present invention provides methods comprising a) analysing a biological sample obtained from a subject to determine the presence of target molecules representative of expression of at least two biomarkers selected from the group listed in Table 1, wherein the biological sample is a breast tissue sample or derivative thereof; and b) comparing the expression levels of the biomarkers determined in (a) with one or more reference values, wherein whether there is a difference in the expression of the biomarkers in the sample from the subject compared to the one or more reference values is indicative of a clinical indication. The invention further provides methods of treatment and kits and assays for use in the methods of the invention.

### FIELD OF THE INVENTION

The present invention relates to methods for diagnosing breast cancer. The invention also relates to methods of assessing the prognosis of a breast cancer and response to treatment. The invention also relates to methods of treating breast cancer. Further, the invention concerns kits and assay devices for use in the methods of the invention.

### BACKGROUND TO THE INVENTION

Breast Cancer is the most common cancer in women (1). Early detection of tumors significantly improves survival rates; more than 90% of women diagnosed with early stage breast cancer survive for at least five years (2). Consequently mammographic screening, by enabling early detection, reduces mortality in women 50-74 years of age although efficacy is more limited for younger women (3), with false positive resulting in overdiagnosis and potentially unnecessary treatment. Other early detection screening methods (e.g., MRI, ultrasonography, clinical and self-breast examination) are inadequate at present to reduce breast cancer mortality. These data underlie an urgent need for improved detection and clinical management of malignant cancers.

The tumor microenvironment is a dominant player of tumor progression and growth. Tumors not only comprise of malignant cells but also a complex stroma in which immune cells are highly represented; cancer cells acquire the ability to “distract and educate” the immune system so that their abnormal proliferation is not detected, but rather promoted (4).

Macrophages are abundant in tumors and in mouse models are derived from circulating monocytes. Experimental models have indicated that Tumor Associated Macrophages (TAMs), whose density correlates with poor prognosis in many human cancers, promote angiogenesis, blunt anti-tumor cytolytic T cell responses, enhance tumor extravasation, dissemination and overt metastasis (5,6). Recent reports have also revealed a positive correlation between circulating monocytes and cancer progression (7-9). In contrast, very little is known on the role of human macrophages in human cancer, and in particular very little is known about the transcriptional programs of mature macrophages once within neoplastic tissues.

### SUMMARY OF THE INVENTION

Accordingly, the present invention provides methods of diagnosing and/or prognosing breast cancer, predicting efficacy of treatment for breast cancer, assessing outcome of treatment for breast cancer or assessing recurrence of breast cancer. The methods comprise the steps of a) analysing a biological sample obtained from a subject to determine the presence of target molecules representative of expression of at least two biomarkers selected from the group listed in Table 1, wherein the biological sample is a breast tissue sample or derivative thereof; and b) comparing the expression levels of the biomarkers determined in (a) with one or more reference values, wherein whether there is a difference in the expression of the biomarkers in the sample from the subject compared to the one or more reference values is indicative of a clinical indication. Preferably the at least two biomarkers will comprise a biomarker for SIGLEC1.

Preferably said clinical indication comprises one or more of the presence or absence of breast cancer in the breast tissue sample from the subject, the receptor status of breast cancer in the tissue sample, for example oestrogen (ER), HER2 and/or progesterone receptor status, tumor grade of breast cancer in the tissue sample, likelihood of metastasis from breast cancer in the tissue sample, likely outcome of treatment of the breast cancer in the subject, likelihood of recurrence of the breast cancer following treatment, an indication of whether the prognosis for the breast cancer and subject is good or poor and/or predicted survival (life expectancy) of the subject.

In preferred methods the reference values will be associated with a particular clinical indication such that a defined difference in the expression of the biomarkers in the sample from the subject compared to the one or more reference values will be indicative of a particular clinical indication. For example, the reference values may be representative of the expression of the same biomarkers in resident macrophages from breast tissue of subjects not having breast cancer, and a diagnosis that the subject has breast cancer will be indicated when there is differential expression of the biomarkers compared to the corresponding biomarker reference values, and/or a diagnosis that the subject does not have breast cancer will be indicated when there is substantially no differential expression.

In also preferred methods, the reference values will be in the form of gene expression signatures corresponding to the biomarker expression levels in macrophages from breast tissue having a known particular clinical indication, and a difference in the expression levels of the biomarkers in the biological sample will be assessed by determining whether said expression levels of the biomarkers of the biological sample correlate with one of the gene expression signatures, thereby stratifying the biological sample breast tissue as being of the same clinical indication as that with which the gene expression signature is correlated. For example, the expression levels of the biomarkers determined in (a) may be compared with one or more gene expression signatures representing gene expression levels of the same biomarkers in macrophages from breast cancer tissue having a good outcome and one or more gene expression signatures representing gene expression levels of the same biomarkers in macrophages from breast cancer tissue having a poor outcome; the biological sample breast tissue will be indicated as being associated with a poor outcome if it stratifies with the poor outcome gene expression signature, and indicated as being associated with a good outcome if it stratifies with the good outcome gene expression signature. The skilled person will appreciate, however, that the gene expression signatures may be representative of any appropriate clinical indications such that correlation determination and stratification can be used to establish with which clinical indication the biological sample breast tissue groups, such that the biological sample breast tissue should be considered as having that clinical indication.

The invention also provides associated methods of treating breast cancer in a subject. The methods comprise the steps of a) analysing a biological sample obtained from a subject to determine the presence of target molecules representative of expression of at least two biomarkers selected from the group listed in Table 1; b) comparing the expression levels of the biomarkers determined in (a) with one or more reference values, and providing the subject with a particular treatment for breast cancer according to whether there is a difference in the expression of the biomarkers in the sample from the subject compared to the one or more reference values. As explained above, in preferred methods the reference values will be associated with a particular clinical indication, such that differential expression analysis may be used to determine whether or not the biological sample breast tissue has that particular indication, and in also preferred methods the reference values will be in the form of gene expression signatures such that correlation and stratification can be used to determine the clinical indication of the biological sample breast tissue; treatment options can be decided according to the determined clinical indication. Preferably the at least two biomarkers will comprise a biomarker for SIGLEC1.

The invention also provides kits for use in the above methods, the kits comprising binding partners capable of binding to target molecules representative of expression of at least two biomarkers selected from the group listed in Table 1. Preferably the kits also comprise indicators capable of indicating when said binding occurs. Preferably the at least two biomarkers will comprise a biomarker for SIGLEC1.

The invention also provides an assay device for use in the above methods, the device comprising: a) a loading area for receipt of a biological sample; b) binding partners specific for target molecules representative of expression of at least two biomarkers selected from the group listed in Table 1; and c) detection means to detect the levels of said target molecules present in the sample. Preferably the at least two biomarkers will comprise a biomarker for SIGLEC1.

### DETAILED DESCRIPTION

The methods of the present invention provide simple tests that may be used in the methods of diagnosing and/or prognosing breast cancer, predicting efficacy of treatment for breast cancer, assessing outcome of treatment for breast cancer or assessing recurrence of breast cancer, and provides methods of treatment using the diagnosis, prognosis, prediction and/or assessment. The kits and devices of the invention are useful for conducting the methods of the invention.

The invention is based on the inventors' significant and surprising finding that tumor associated macrophages (TAMs) exhibit specific tissue and cancer transcriptional profiles; they have identified 37 genes that are differentially expressed in breast TAMs and able to predict properties of the breast cancer, recurrence, and overall survival in publicly available breast cancer datasets. Thus human TAM-specific transcriptional changes can be used in diagnosis and as a prognostic factor for breast cancer progression.

The inventors' are therefore able to provide herein specific biomarkers that show differential expression in breast cancer samples and that segregate breast cancer samples obtained from subjects with a poor prognosis for breast cancer from breast cancer samples obtained from subjects with a good prognosis for breast cancer. The inventors' work in identifying these clinically useful biomarkers has led them to the instant invention, involving new methods of cancer diagnosis, prognosis and prediction. The ability to stratify and identify breast cancers in this way means that the clinician is better able to tailor treatment options for each subject, according to the specific clinical indications of their breast cancer, and also better able to discuss the likely disease course and outcome with the subject. Advantageously, the inventors have found that the biomarkers can be usefully detected and analyzed in total transcriptomes of breast cancer to predict outcome and/or treatment response, for example, as well as being usefully detected and analyzed using immunohistochemistry or other in situ methods on breast cancer tissue. Particularly preferred methods for using in detecting and analyzing biomarkers in accordance with the invention include spatial and single cell transcriptomics.

Of course, in some embodiments the methods of the invention may be used in combination with other methods of detecting, diagnosing, prognosing and/or treating cancer, in which case the combination may advantageously increase specificity and sensitivity compared to use of the other methods on their own, and allow the prioritization of the identification, follow-up and treatment of those most likely to require further and more aggressive treatment. Similarly, the methods may advantageously be used to predict the outcome of patients that are not correctly classified by preliminary and/or current routine methods, for example patients with samples that fall within an intermediate diagnosis group and patients with ER negative (ER−) tumors. Currently, techniques such as MammaPrint and Oncotype Dx are used primarily and are most accurate for ER+/HER2− tumors, so that the methods disclosed herein may complement such techniques by providing predictions for ER− samples.

Thus methods of the invention may also allow patients with aggressive breast cancer to be identified swiftly, and guide medical staff to commence appropriate treatment promptly.

In addition, since the biomarkers of the invention are derived from expression patterns in the stromal tissue associated with the malignant cells, rather than being derived from the expression in the malignant (e.g epithelial) cells, the methods of the invention may still be used after treatment or surgery intended to reduce or eliminate the cancerous cell mass.

In order to assist the understanding of the present invention, certain terms used herein will now be further defined, and more generally further details of the invention will be given, in the following paragraphs.

### Biological Sample

The biomarker expression levels are analysed in a biological sample obtained from a subject. The biological sample is preferably a tissue sample or a derivative thereof, preferably a breast tissue sample or a derivative thereof, and further preferably the tissue sample will be breast tissue removed during diagnostic, for example core needle biopsy or fine needle aspiration, preventative, curative, palliative and/or reconstructive surgery. Preferably the tissue will have been removed from a subject having, or suspected of having, cancer, for example in, or in the vicinity of, the removed tissue.

Preferably the biological sample will comprise, or substantially consist of, stromal tissue that is or was adjacent to the cancerous cells. The inventors have surprisingly found that the methods of the invention may be carried out on the stromal tissue that supports, or has supported, the cancerous cells. Thus, the methods of the invention may involve using stromal tissue that has been removed from a site where cancerous cells are suspected or have been detected in the past, and carrying out methods of the invention on the stromal tissue to obtain a diagnosis, prognosis, or prediction regarding the cancer. An attempt may have been made to remove, e.g. surgically, or destroy, e.g. chemically and/or using radiation, the cancerous cells and the methods of the invention may provide an analysis as to how successful that removal (i.e. treatment) has been and what the likely outcome will be for the subject; that treatment may have immediately preceded the removal of the stromal tissue for use in the methods of the invention, or alternatively the stromal tissue may be removed a significant period of time after the treatment, for example weeks, months, or years after the treatment, in which case the methods of the invention may be used, for example, to monitor the likely recurrence or metastasis of the cancer.

Methods of the invention may involve detecting expression levels in tissue samples in which the target molecules have been labelled, for example using immunohistochemistry (IHC), preferably multiplex IHC (mIHC), for mass spectrometry or spatial transcriptomics, or fluorescence in situ hybridization (FISH) to detect RNA molecules (RNA FISH). Such methods for labelling target molecules may be included in the methods of the invention, and associated reagents may be included in the kits and devices for use in the methods of the invention, for example reagents required to carry out IHC, mIHC, and/or RNA FISH. Preferably there will be no artificial enrichment of macrophages in the tissue sample prior to the analysis, such that up to 60% of the cells in the tissue sample may be macrophages, for example between 5 and 55% of cells in the tissue. In particularly preferred methods, IHC will be performed to detect and quantify target molecules present in tissue that has been removed before therapeutic treatment and in corresponding tissue that has been removed after therapeutic treatment, with a comparison made of the concentration or number of target molecules present in each tissue sample, in order to detect changes in expression in response to treatment.

Expression levels may be selectively detected in macrophages of the biological sample. Preferably the biological sample, or part thereof, in which the levels are detected will be enriched for macrophages or may substantially consist of macrophages. Thus the sample may be enriched for macrophages or may substantially consist of macrophages, for example at least 75% of the cells in the biological sample may be macrophages, for example 80%, 85%, 90%, 95%, 96%, 97%, 97.5% 98%, 99%, 99.5% or 99.8% of the cells in the sample will be macrophages. It is particularly preferred that at least 97% of the cells in the sample will be macrophages.

Preferably the expression levels of the biomarkers are selectively detected in macrophages of the biological sample. Therefore it is particularly preferred that the biological sample, or part thereof, in which the levels are detected will be enriched for macrophages or may substantially consist of macrophages. Suitable methods for artificially enriching samples for macrophages are known to those of skill in the art, for example using FACS sorting or commercially available kits like magnetic cell isolation kits (e.g. by Miltenyi Biotec), which may make use of selective antibodies to CD163, CD68, CD169 coupled to magnetic beads, or physical separations such as using percoll, CyTOF, or FACS isolation. Such methods for enrichment may be included in the methods of the invention, and associated reagents may be included in the kits and devices for use in the methods of the invention.

Alternatively or additionally, the step of analysing the levels of the biomarkers may specifically target the macrophages for that analysis. For example, the analysis may take place on the magnetic beads to which the macrophages specifically attach, such that even though the biological sample may be breast biopsy tissue for example, the expression levels analysed substantially correspond only to the levels in the macrophages of the sample, or the methods may make use of FACS isolation, CyTOF, targeted in situ RNA or protein methods in tissue to target analysis at expression by macrophages in the tissue. Thus it may not be necessary to enrich for macrophages when preparing a sample for use in the methods of the invention.

It is preferred that the sample will be enriched for macrophages, or the step of analyzing the levels of the one or more biomarkers will specifically target the macrophages, for example when the expression levels of SIGLEC1 are to be analyzed.

Alternatively or additionally, it may be preferable to take the numbers of macrophages into account when carrying out the analysis of the expression levels of the one or more biomarkers in the sample, so that the analysis will indicate, for example a difference in expression levels of at least one biomarker that may be due to a difference in the relative number of macrophages in the tissue, and/or the analysis may include, for example, an indication of the number or concentration of macrophages present in the sample (for example, whether the tissue from which the sample was taken is relatively enriched for macrophages or not, according to how many macrophages there are in a particular amount of tissue), the expression levels per macrophage, and/or how many of the macrophages present in the sample express the biomarker of interest. Thus in one embodiment the analysis may take place on a section of tissue, and the tissue may be stained, using one or more biomarkers of the invention or otherwise, such that the macrophages present in the tissue are identifiable and the expression levels associated with those macrophages can be assessed according to how many macrophages there are expressing each biomarker in a particular area or volume of tissue. In this way, it is possible to identify increases in specific populations of macrophages, which increases in population correlate with a poor outcome for the subject from whom the tissue was taken.

Particularly where there is no artificial enrichment for macrophages in the sample, the analysis of biomarker levels may reflect the number of macrophages present in the tissue; for example, an increase in the level of one or more biomarkers in the tissue may, at least in part, be due to a larger number of macrophages (TAMs) in the tissue when compared to the number of resident macrophages in an equivalent amount of normal tissue. Thus it may not be necessary to enrich for macrophages when preparing a sample for use in the methods of the invention. The method may involve obtaining a sample of biological material from the subject, or it may be performed on a pre-obtained sample, e.g. one which has been obtained previously for this or other clinical purposes. Similarly, the biological sample obtained from the subject may be processed before use in methods of the invention, for example to enrich for macrophages, and/or the methods of the invention may include suitable processing steps to enrich for or identify macrophages in the sample, for example through the use of selective magnetic separation systems such as those mentioned above.

In some embodiments the methods of the present invention may make use of multiple biological samples taken from a subject to determine the expression level of one or more biomarkers.

### A Subject

In the context of the methods and medical uses of the present invention, a subject may be anyone requiring the diagnosis, prognosis and/or treatment for breast cancer. Suitably the subject will be a mammal, preferably a primate and further preferably a human subject. The subject may be of any sex, for example female or male.

As mentioned elsewhere in the specification, the subject may present with symptoms consistent with breast cancer and/or they may have already undergone tests that have suggested that they have breast cancer. Breast tissue may be removed from such subjects during diagnostic, curative, palliative and/or reconstructive surgery as described above. For such a subject, the removed breast tissue may be used in a method of the invention to indicate, for example, the presence of breast cancer, and optionally the grade and/or ER status of any breast cancer present, as explained further below.

Alternatively, the subject may appear to be asymptomatic. Suitably an asymptomatic subject may be a subject who is believed to be at elevated risk of having cancer, for example breast cancer. Such an asymptomatic subject may be one who has a family history of early-onset of cancer, such as breast cancer, or who has an increased risk of an age-related cancer, such as breast cancer. For example, the subject may be a subject considered to be at increased risk of developing breast cancer who has a prophylactic mastectomy, and the breast tissue removed may be used in a method described herein in order to check for the presence of breast cancer.

### Levels of Biomarkers

Methods of the invention involve looking at the expression levels of biomarkers selected from the genes of Table 1, i.e. biomarkers corresponding to the genes listed in Table 1. The methods involve looking at the levels of at least two biomarkers corresponding to genes in the list, for example at least 3, 4, 5, 8, 10, 12, 15, 18, 20, 21, 23, 25, 28, 30, 32, 35, 36, or 37 biomarkers corresponding to genes in the list of Table 1. Preferably the methods involve looking at the expression levels of biomarkers corresponding to at least 5 genes in the list of Table 1, at least 15, at least 21, or at least 30 genes in the list of Table 1. The methods may comprise looking at the expression levels of all the biomarkers in Table 2, and optionally all the biomarkers in Table 1. Determining the abundance of larger numbers of biomarkers can be preferable as it can allow for a more reliable or powerful test. This can occur for many reasons, e.g. because combining information about a plurality of markers reduces the risk that a single biomarker might unduly skew the result due to an altered abundance of an unrelated cause, and because changes in a broader pattern of abundance levels can be highly informative. The kits and devices of the invention correspondingly provide binding partners for looking at the levels of two biomarkers in the list, for example at least 3, 4, 5, 8, 10, 12, 15, 18, 20, 21, 23, 25, 28, 30, 32, 35, 36, or 37 biomarkers, in accordance with the methods of the invention disclosed herein.

TABLE 1
37 gene TAM metagene signature

| | | Relative |
|---|---|---|
| Gene Name | Gene Full Name | Expression |
| IRF8 | Interferon regulatory factor 8 | Increased |
| CCL2 | Chemokine (C—C Motif) ligand 2 | Increased |
| C1QC | Complement component 1qC | Increased |
| GBP5 | Guanylate binding protein 5 | Increased |
| HCST | Hematopoietic cell signal transducer | Increased |
| LILRB4 | Leukocyte immunoglobulin-like receptor | Increased |
| | subfamily B member 4 | |
| AIF1 | Allograft inflammatory factor-1 | Increased |
| PSMB9 | Proteasome subunit beta type-9 | Increased |
| GBP4 | Guanylate binding protein 4 | Increased |
| GBP1 | Guanylate binding protein 1 | Increased |
| HLA-DOA | HLA Class II histocompatibility antigen, | Increased |
| | DO alpha chain | |
| C1QA | Complemen component 1qA | Increased |
| CCL4 | Chemokine (C—C Motif) ligand 4 | Increased |
| NCF1C | Putative neutrophil cytosol factor 1C | Increased |
| LAP3 | Leucine aminopeptidase 3 | Increased |
| TNFAIP3 | Tumor necrosis factor, alpha-induced | Increased |
| | protein 3 | |
| ITGB2 | Integrin beta chain-2 | Increased |
| LAIR1 | Leukocyte-associated immunoglobulin- | Increased |
| | like receptor 1 | |
| FOLR2 | Folate receptor beta | Increased |
| CD83 | Cluster of differentiation 83 | Increased |
| SIGLEC1 | Sialoadhesin | Increased |
| TCN2 | Transcobalamin II | Increased |
| PLTP | phospholipid transfer protein | Increased |
| C1QB | Complement component 1qB | Increased |
| DOK2 | Docking protein 2 | Increased |
| GIMAP6 | GTPase IMAP family member 6 | Increased |
| CD40 | Cluster of differentiation 40 | Increased |
| CCL3 | Chemokine (C—C Motif) ligand 3 | Increased |
| CCL8 | Chemokine (C—C Motif) ligand 8 | Increased |
| FCN1 | Ficolin-1 | Increased |
| CD4 | Cluster of differentiation 4 | Increased |
| VAV1 | Proto-oncogene vav | Increased |
| TLR7 | Toll-like receptor 7 | Increased |
| FGD2 | FYVE, RhoGEF and PH domain- | Increased |
| | containing protein 2 | |
| LST1 | Leukocyte-specific transcript 1 protein | Increased |
| VSIG4 | V-set and immunoglobulin domain | Increased |
| | containing 4 | |
| CLEC7A | C-type lectin domain family member A | Increased |

Preferably the biomarkers of the methods, kits and devices of the invention will comprise at least 5, 10, 15, 20, or all 21, of the biomarkers in Table 2, i.e. biomarkers corresponding to the genes listed in Table 2.

TABLE 2
Preferred subset of biomarkers of Table 1.

| | | Relative |
|---|---|---|
| Gene Name | Gene Full Name | Expression |
| C1QC | Complement component 1qC | Increased |
| GBP5 | Guanylate binding protein 5 | Increased |
| LILRB4 | Leukocyte immunoglobulin-like receptor | Increased |
| | subfamily B member 4 | |
| PSMB9 | Proteasome subunit beta type-9 | Increased |
| GBP4 | Guanylate binding protein 4 | Increased |
| GBP1 | Guanylate binding protein 1 | Increased |
| C1QA | Complement component 1qA | Increased |
| CCL4 | Chemokine (C—C Motif) ligand 4 | Increased |
| NCF1C | Putative neutrophil cytosol factor 1C | Increased |
| TNFAIP3 | Tumor necrosis factor, alpha-induced | Increased |
| | protein 3 | |
| ITGB2 | Integrin beta chain-2 | Increased |
| LAIR1 | Leukocyte-associated immunoglobulin- | Increased |
| | like receptor 1 | |
| CD83 | Cluster of differentiation 83 | Increased |
| SIGLEC1 | Sialoadhesin | Increased |
| PLTP | phospholipid transfer protein | Increased |
| C1QB | Complement component 1qB | Increased |
| CCL3 | Chemokine (C—C Motif) ligand 3 | Increased |
| CCL8 | Chemokine (C—C Motif) ligand 8 | Increased |
| CD4 | Cluster of differentiation 4 | Increased |
| VAV1 | Proto-oncogene vav | Increased |
| VSIG4 | V-set and immunoglobulin domain | Increased |
| | containing 4 | |

In particularly preferred methods, the biomarkers will comprise SIGLEC1. For example the biomarkers may be SIGLEC1 and at least 1, 2, 3, 5, 8, 10, 15, 20, 23, 25, 28, 30, 32, or 35 other biomarkers selected from Table 1, and further preferably SIGLEC1 and at least 1, 2, 3, 5, 8, 10, 15, or 20 other biomarkers selected from Table 2. Preferably, one of the other biomarkers will be CCL8 such that expression levels of SIGLEC1 and CCL8 are determined, optionally as well as at least 1, 2, 3, 5, 8, 10, 15, 20, 23, 25, 28, 30, 32, or 34 other biomarkers selected from Table 1, and further optionally expression levels of SIGLEC1 and CCL8 are determined as well as at least 1, 2, 3, 5, 8, 10, 15, or 18 other biomarkers selected from Table 2. The biomarkers of methods of the invention may consist of the biomarkers in Table 2. The biomarkers of methods of the invention may consist of the biomarkers in Table 1.

It will be apparent to the skilled person that the abovementioned selections and combinations of biomarkers from Tables 1 and 2 represent various minimal marker sets, and additional biomarkers, whether selected from the list of Table 1 or not, can also be included. Alternatively, in some methods, kits and devices of the invention biomarkers selected from Table 1, and optionally Table 2, may be the only biomarkers for which the expression levels are assessed. However, the methods, kits and devices may also provide for the assessment of control target molecules in the biological sample, for example control target molecules that display substantially constant abundance irrespective of the abundance of the biomarkers of Table 1 and/or the clinical status of the biological sample, where the assessment of the control target molecules allow for the accuracy of the assessment mechanism to be tested.

The invention involves assessing changes in levels for biomarkers, and in preferred embodiments this change is typically differentially upwards for the genes in Table 1 and 2, in subjects having a particular clinical indication, preferably a diagnosis that breast cancer is present. The biomarkers were found to be overexpressed in Breast TAMs, compared to resident macrophages, and found to be highly expressed in the most aggressive breast cancer subtypes and enriched in a CSF1-high group that has been previously associated with higher tumor grade, decreased expression of estrogen and progesterone receptor, and higher mutation rate. The biomarkers were also found to be associated with shorter disease-specific survival. Therefore, it will be understood that although relative expression of the biomarkers will depend on the reference values used, generally higher relative expression of the biomarkers, compared to relative expression in resident macrophages of normal breast tissue or relative expression in TAMs of breast tissue associated with low grade cancer with good prognosis, will be indicative of a poorer outcome, for example the presence of cancer, the presence of high grade cancer, a poorer likely survival rate, etc, as explained further below.

Throughout, biomarkers in the biological sample(s) from the subject are said to be expressed at different levels, or differentially expressed, where they are significantly up- or down-regulated compared to a reference value of expression. Depending on the individual biomarker, a breast cancer diagnosis may be given from a biological sample based on either an increase or decrease in expression level, optionally scaled in relation to sample mean and sample variance, relative to those of subjects not having breast cancer or one or more reference values. Clearly, variation in the sensitivity of individual biomarkers, subject and samples mean that different levels of confidence are attached to each biomarker. Biomarkers of the invention are said to be significantly upregulated when, optionally after scaling of biomarker expression levels in relation to sample mean and sample variance, they exhibit at least a 1.5-fold change, preferably a 2-fold change, compared with subjects not having cancer or one or more reference values (i.e. a log₂ fold change of greater than 0.58 or less than −0.58, preferably greater than +1 or less than −1). Preferably biomarkers will exhibit a 3-fold change or more compared with the reference value. More preferably biomarkers of the invention will exhibit a 4-fold change or more compared with the reference value. That is to say, in the case of increased expression level (up-regulation relative to reference values), the biomarker level will be more than double that of the reference value. Preferably the biomarker level will be more than 3 times the level of the reference value. More preferably, the biomarker level will be more than 4 times the level of the reference value.

The term “reference value” may refer to a pre-determined reference value, for instance specifying a confidence interval or threshold value for a clinical indication, for example a diagnosis that breast cancer is present or for prediction of the susceptibility of a subject to treatment and/or recurrence. Alternatively, the reference value may be derived from the expression level of a corresponding biomarker or biomarkers in a ‘control’ biological sample, for example a positive (breast tissue macrophages from a patient having a breast cancer diagnosis and/or not being susceptible to treatment and/or having a poor outcome) or negative (breast tissue macrophages from a patient not diagnosed with breast cancer or a patient diagnosed with breast cancer that proved susceptible to treatment or a patient diagnosed with breast cancer who had a successful outcome) control. Furthermore, the reference value may be an ‘internal’ standard or range of internal standards, for example a known concentration of a protein, transcript, label or compound within the sample. Alternatively, the reference value may be an internal technical control for the calibration of expression values or to validate the quality of the sample or measurement techniques. This may involve a measurement of one or several transcripts within the sample which are known to be constitutively expressed or expressed at a known level (e.g. an invariant level). Accordingly, it would be routine for the skilled person to apply these known techniques alone or in combination in order to quantify the level of biomarker in a sample relative to standards or other transcripts or proteins or in order to validate the quality of the biological sample, the assay or statistical analysis.

In preferred methods of the invention the reference values correspond to the levels of the same biomarkers in resident macrophages from tissue, preferably breast tissue, not associated with breast cancer i.e. from samples from subjects not having breast cancer. Thus the reference values may be representative of corresponding values in subjects not having breast cancer. A comparison of the expression levels of the biomarkers in the biological sample from the subject with the reference values corresponding to those from a subject not having breast cancer will therefore show whether there is a difference in expression of the biomarkers relative to the resident macrophage samples, and a difference in expression of the biomarkers, as explained further below, will be indicative of a diagnosis that there is breast cancer present in the biological sample breast tissue or that there is, or was, breast cancer present in tissue adjacent to the tissue of the biological sample. The skilled person will understand that since the expression of the genes of Table 1 and 2 were found to be overexpressed in Breast TAMs, compared to the expression in resident macrophages not associated with breast cancer, then when the reference values are representative of corresponding values in subjects not having breast cancer, the biomarkers will be overexpressed in samples from subjects having breast cancer and/or a poorer prognosis.

Alternatively the reference values may correspond to the levels of the biomarkers in samples from subjects who had been diagnosed with breast cancer and for whom one or more of the clinical details regarding the breast cancer are known, such as the outcome of treatment of the cancer, a receptor status such as ER status, and/or the recurrence status. Thus the reference values may be representative of corresponding values in subjects who have been successfully treated for breast cancer, in subjects who have been unsuccessfully treated for breast cancer, and/or in subjects previously successfully treated for whom the breast cancer has returned. Similarly, in some methods involving providing a prognosis for a subject and/or predicting a subject's response to (a particular) treatment, the reference values may correspond to the levels of the biomarkers in samples from subjects with a particular known prognosis or response to a particular treatment.

In preferred methods of the invention the reference values correspond to one or more gene expression signatures each derived from one or more patients having a particular clinical indication. Thus a gene expression signature as used herein refers to a biomarker gene expression pattern which is characteristic of, or correlated with, resident macrophages from normal breast tissue or TAMs from breast cancers having a particular clinical indication, such as a particular receptor status, a particular grade of breast cancer (e.g. grade I, II, or Ill, or combinations thereof), a poor prognosis/outcome, a good prognosis/outcome, good response to treatment, poor response to treatment, a poor survival rate, a good survival rate, a generally good outcome, a generally intermediate outcome, a generally poor outcome, the presence or absence of local and/or distant metastasis, and recurrence or no recurrence. The expression levels of the biomarkers in the biological sample will be compared with the expression levels of the same biomarkers in the gene expression signatures to determine if the expression levels of the biological sample correlate to a gene expression signature, such that the subject and associated breast tissue stratify with the group of patients with the clinical indication of that gene expression signature.

The skilled person will understand that a gene expression signature can be established using patient samples of breast tissue where the clinical indications are known. Gene expression signatures can include relative or absolute expression levels of biomarkers, and can be compared to sample biomarker expression levels which are correspondingly also either relative or absolute. The skilled person will appreciate that gene expression signatures may be built using any number of breast tissue samples from patients having the same clinical indication, although the gene expression signatures will generally become more stable as increasing numbers of patient samples are used to build them. Also, as increasing numbers of patient samples of are used, the skilled person will understand that it will be possible to assess how much weight should be given to the expression of each biomarker within the gene expression signature, for example according to the amount of variation that is shown by that biomarker across the patient samples used to generate the signature. Methods for generating and optimising, for example to achieve the desired sensitivity and specificity, such gene expression signatures, and using them for comparisons, correlations and stratifications, are well known in the art.

Preferably the subjects used to generate the reference values will be “matched” to some extent with those providing the biological sample. For example, if the subject providing the sample is a female suspected of having breast cancer then preferably the subjects providing the reference values will also be female. Similarly, if the subject providing the sample is an adolescent female suspected of having cancer then preferably the subjects providing the reference values will also be adolescent females. Thus the subjects providing the samples to which the reference values correspond may be “matched” according to sex and/or age. Alternatively the subjects providing the samples to which the reference values correspond may comprise a range of ages and/or sexes. Similarly, preferably the samples used to generate the reference values will be processed in the same way as the interrogated biological samples, according to the methods of the invention, for example with the same methods used to enrich for macrophages and/or interrogate the expression levels of the biomarkers.

It will be apparent to the skilled person that there is considerable freedom to interpret and process data obtained by the methods of the present invention, and how to interpret or act upon the results. It may be desirable, for example, to prioritise sensitivity or specificity, or to optimise positive predictive value or negative predictive value. Depending on the context in which the method is performed, the skilled person can therefore select appropriate methods of interpreting the results. For example, the results for different markers can be weighted in different ways, different threshold abundance levels can be applied, various statistical analyses can be applied, and one or more indicators of a potentially dubious test result can be determined.

The person skilled in the art is free to formulate a wide range of calculations, e.g. via suitable algorithms, in order to obtain a desired result from processing data obtained from the methods of the present invention. There involves routine application of well-known statistical analysis techniques.

Interferon regulatory factor 8 (IRF8) also known as interferon consensus sequence-binding protein (ICSBP), is a protein that in humans is encoded by the IRF8 gene. IRF8 is a transcription factor that plays critical roles in the regulation of lineage commitment and in myeloid cell maturation including the decision for a common myeloid progenitor (CMP) to differentiate into a monocyte precursor cell. The inventors have surprisingly found that this gene is significantly overexpressed in TAMs found in breast cancer, compared to the expression in resident macrophages not associated with breast cancer. Therefore in methods of the invention in which the expression levels of IRF8 are analysed, it is preferred that significant up-regulation of the expression level of IRF8 in a sample from a subject is associated with the subject having a particular clinical indication, preferably a diagnosis that breast cancer is present. For example, a log₂ fold change of at least 0.6, for example a log 2 fold change of at least 1, 1.1, 1.2, 1.3, 1.4, 1.5, 1.6, 1.7, 1.8, 1.9, or 2.0, in a sample compared to one or more reference values may be indicative of the subject having a having a particular clinical indication, preferably a diagnosis of breast cancer. The skilled person will appreciate that the relative expression levels of IRF8 will depend on the reference values used in the comparison; however, in preferred methods the reference values will correspond to the levels of the biomarkers in resident macrophages from subjects not having cancer, and a log₂ fold change of at least 1, preferably at least 1.25, in expression levels of IRF8 will be indicative of a diagnosis that breast cancer is present.

The chemokine (C-C motif) ligand 2 (CCL2) is also referred to as monocyte chemoattractant protein 1 (MCP1) and small inducible cytokine A2. CCL2 is a small cytokine that belongs to the CC chemokine family. CCL2 recruits monocytes, memory T cells, and dendritic cells to the sites of inflammation produced by either tissue injury or infection. The inventors have surprisingly found that this gene is significantly overexpressed in TAMs found in breast cancer, compared to the expression in resident macrophages not associated with breast cancer. Therefore in methods of the invention in which the expression levels of CCL2 are analysed, it is preferred that significant up-regulation of the expression level of CCL2 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, 3.4, 3.5, 3.6, 3.7, 3.8, 3.9, or 4.0 in a sample compared to one or more reference values may be indicative of the subject having a particular clinical indication, preferably a diagnosis that breast cancer is present. The skilled person will appreciate that the relative expression levels of CCL2 will depend on the reference values used in the comparison; however, in preferred methods the reference values will correspond to the levels of the biomarkers in resident macrophages from subjects not having cancer, and a log₂ fold change of at least 2, preferably at least 2.5, 3.0, 3.5, or 4.0, in expression levels of CCL2 will be indicative of a diagnosis that breast cancer is present.

C1QC (complement C1q C chain) is located on chromosome 1 and it encodes the C-chain polypeptide of serum complement subcomponent C1q, which associates with C1r and C1s to yield the first component of the serum complement system. The inventors have surprisingly found that this gene is significantly overexpressed in TAMs found in breast cancer, compared to the expression in resident macrophages not associated with breast cancer. Therefore in methods of the invention in which the expression levels of C1QC are analysed, it is preferred that significant up-regulation of the expression level of C1QC in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, 3.4, 3.5, 3.6, 3.7, 3.8, 3.9, or 4.0 in a sample compared to one or more reference values may be indicative of the subject having a particular clinical indication, preferably a diagnosis that breast cancer is present. The skilled person will appreciate that the relative expression levels of C1QC will depend on the reference values used in the comparison; however, in preferred methods the reference values will correspond to the levels of the biomarkers in resident macrophages from subjects not having cancer, and a log₂ fold change of at least 2, preferably at least 2.5, 3.0, 3.5, or 4.0, in expression levels of C1QC will be indicative of a diagnosis that breast cancer is present.

C1QA (complement C1q A chain) is located on chromosome 1 and it encodes the A-chain polypeptide of serum complement subcomponent C1q, which associates with C1r and C1s to yield the first component of the serum complement system. The inventors have surprisingly found that this gene is significantly overexpressed in TAMs found in breast cancer, compared to the expression in resident macrophages not associated with breast cancer. Therefore in methods of the invention in which the expression levels of C1QA are analysed, it is preferred that significant up-regulation of the expression level of C1QA in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, 3.4, 3.5, 3.6, 3.7, 3.8, 3.9, or 4.0 in a sample compared to one or more reference values may be indicative of the subject having a particular clinical indication, preferably a diagnosis that breast cancer is present. The skilled person will appreciate that the relative expression levels of C1QA will depend on the reference values used in the comparison; however, in preferred methods the reference values will correspond to the levels of the biomarkers in resident macrophages from subjects not having cancer, and a log₂ fold change of at least 2, preferably at least 2.5, 3.0, 3.5, or 4.0, in expression levels of C1QA will be indicative of a diagnosis that breast cancer is present.

C1QB (complement C1q B chain) is located on chromosome 1 and it encodes the B-chain polypeptide of serum complement subcomponent C1q, which associates with C1r and C1s to yield the first component of the serum complement system. The inventors have surprisingly found that this gene is significantly overexpressed in TAMs found in breast cancer, compared to the expression in resident macrophages not associated with breast cancer. Therefore in methods of the invention in which the expression levels of C1QB are analysed, it is preferred that significant up-regulation of the expression level of C1QB in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, 3.4, 3.5, 3.6, 3.7, 3.8, 3.9, or 4.0 in a sample compared to one or more reference values may be indicative of the subject having a particular clinical indication, preferably a diagnosis that breast cancer is present. The skilled person will appreciate that the relative expression levels of C1QB will depend on the reference values used in the comparison; however, in preferred methods the reference values will correspond to the levels of the biomarkers in resident macrophages from subjects not having cancer, and a log₂ fold change of at least 2, preferably at least 2.5, 3.0, 3.5, or 4.0, in expression levels of C1QB will be indicative of a diagnosis that breast cancer is present.

CD83 (CD83 molecule) is located on chromosome 6 and it is a single-pass type I membrane protein and member of the immunoglobulin superfamily of receptors. The inventors have surprisingly found that this gene is significantly overexpressed in TAMs found in breast cancer, compared to the expression in resident macrophages not associated with breast cancer. Therefore in methods of the invention in which the expression levels of CD83 are analysed, it is preferred that significant up-regulation of the expression level of CD83 in a sample from a subject is associated with the subject having a particular clinical indication, preferably a diagnosis that breast cancer is present. For example, a log₂ fold change of at least 0.6, for example a log₂ fold change of at least 1, 1.5, 1.8, 1.9, 2.0, 2.1, 2.2, 2.3, 2.4, 2.5, 2.6, or 2.7, in a sample compared to one or more reference values may be indicative of the subject having a particular clinical indication, preferably a diagnosis that breast cancer is present. The skilled person will appreciate that the relative expression levels of CD83 will depend on the reference values used in the comparison; however, in preferred methods the reference values will correspond to the levels of the biomarkers in resident macrophages from subjects not having cancer, and a log₂ fold change of at least 1.0, preferably at least 1.5 or 2.0, in expression levels of CD83 will be indicative of a diagnosis that breast cancer is present.

CLEC7A (C-type lectin domain containing 7A) is located on chromosome 12 and it encodes a member of the C-type lectin/C-type lectin-like domain (CTL/CTLD) superfamily. The inventors have surprisingly found that this gene is significantly overexpressed in TAMs found in breast cancer, compared to the expression in resident macrophages not associated with breast cancer. Therefore in methods of the invention in which the expression levels of CLEC7A are analysed, it is preferred that significant up-regulation of the expression level of CLEC7A in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, 3.4, 3.5, 3.6, 3.7, 3.8, 3.9, or 4.0 in a sample compared to one or more reference values may be indicative of the subject having a particular clinical indication, preferably a diagnosis that breast cancer is present. The skilled person will appreciate that the relative expression levels of CLEC7A will depend on the reference values used in the comparison; however, in preferred methods the reference values will correspond to the levels of the biomarkers in resident macrophages from subjects not having cancer, and a log₂ fold change of at least 2, preferably at least 2.5, 3.0, 3.5, or 4.0, in expression levels of CLEC7A will be indicative of a diagnosis that breast cancer is present.

ITGB2 (integrin subunit beta 2) is located on chromosome 21 and it encodes an integrin beta chain, which combines with multiple different alpha chains to form different integrin heterodimers. The inventors have surprisingly found that this gene is significantly overexpressed in TAMs found in breast cancer, compared to the expression in resident macrophages not associated with breast cancer. Therefore in methods of the invention in which the expression levels of ITGB2 are analysed, it is preferred that significant up-regulation of the expression level of ITGB2 in a sample from a subject is associated with the subject having a particular clinical indication, preferably a diagnosis that breast cancer is present. For example, a log₂ fold change of at least 0.6, for example a log₂ fold change of at least 1, 1.5, 2.0, 2.1, 2.2, 2.3, 2.4, 2.5, 2.6, 2.7, 2.8, 2.9, 3.0, or 3.1, in a sample compared to one or more reference values may be indicative of the subject having a particular clinical indication, preferably a diagnosis that breast cancer is present. The skilled person will appreciate that the relative expression levels of ITGB2 will depend on the reference values used in the comparison; however, in preferred methods the reference values will correspond to the levels of the biomarkers in resident macrophages from subjects not having cancer, and a log₂ fold change of at least 1.5, preferably at least 2.0, 2.5, or 3.0, in expression levels of ITGB2 will be indicative of a diagnosis that breast cancer is present.

SIGLEC1 (sialic acid binding Ig like lectin 1, also called CD169) is located on chromosome 20 and it encodes a member of the immunoglobulin superfamily. The inventors have surprisingly found that this gene is significantly overexpressed in TAMs found in breast cancer, compared to the expression in resident macrophages not associated with breast cancer. Therefore in methods of the invention in which the expression levels of SIGLEC1 are analysed, it is preferred that significant up-regulation of the expression level of SIGLEC1 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 3.0, 3.5, 4.0, 4.5, 5.0, 5.1, 5.2, 5.3, 5.4, 5.5, 5.6, 5.7, 5.8, 5.9, or 6.0 in a sample compared to one or more reference values may be indicative of the subject having a particular clinical indication, preferably a diagnosis that breast cancer is present. The skilled person will appreciate that the relative expression levels of SIGLEC1 will depend on the reference values used in the comparison; however, in preferred methods the reference values will correspond to the levels of the biomarkers in resident macrophages from subjects not having cancer, and a log₂ fold change of at least 2, preferably at least 2.5, 3.0, 3.5, 4.0, 4.5, 5.0 or 5.5, in expression levels of SIGLEC1 will be indicative of a diagnosis that breast cancer is present. It is particularly preferred that the group of biomarkers selected from Table 1 and/or Table 2 in accordance with the methods, kits and assay disclosed herein will comprise SIGLEC1.

In the experiments described below, the inventors show that SIGLEC1 expression is increased in TAMs, particularly TAMs associated with a poor prognosis. This has been shown by the inventors at both the RNA and the protein level. Therefore the skilled person will appreciate that SIGLEC1 expression can similarly be analyzed in the methods of the invention by looking at nucleic acid and/or protein levels. The skilled person will also appreciate, and it is illustrated below, that when nucleic acid levels of SIGLEC1 are analyzed it is preferable to analyze the expression in samples enriched for macrophages, and/or to specifically analyze the expression in the macrophages of the sample as explained above, whilst when protein levels of SIGLEC1 are analyzed this is less important so that it is preferable to analyze samples, for example histological sections, of tissue that retains its cell ratio and structure.

Suitably, the methods may involve analyzing the number of cells (macrophages) in the tissue sample that express SIGLEC1 protein, i.e. that are CD169 positive (CD169+). Preferably in such methods, the reference value will be 5, 10, 15, 20, 25, 30, 35, 40, 45, 50, 60, 70, or 75 CD169+ cells per mm² of tissue section, such that an expression level of greater than 5, 10, 15, 20, 25, 30, 35, 40, 45, 50, 60, 70, or 75 CD169+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. Further preferably in such methods, the reference value will be 25 CD169+ cells per mm² of tissue section, such that an expression level of greater than 25 CD169+ cells per mm² of tissue section will be indicative of the subject having a particular clinical indication, preferably a diagnosis that cancer is present, an indication that metastasis is likely, an indication that recurrence is likely and/or an indication of a poor prognosis or prediction. Generally the greater the increase in the number of CD169+ cells per mm² of tissue section, compared to reference values based on similar analysis of normal tissue or cancer tissue having a good prognosis, the greater the likelihood that the subject has a particular clinical indication, preferably a diagnosis that cancer is present, an indication that metastasis is likely, an indication that recurrence is likely and/or an indication of a poor prognosis or prediction, i.e. higher numbers of CD169+ cells per mm² of tissue section are associated with a poorer prognosis. When analysis of the number of cells per mm² of tissue section is carried out in this way, it is preferred that the expression level is calculated based on the mean value of the number of positive cells in a particular size of tissue section, such as 5, 10, 15, or 20 mm² of tissue section, since the skilled person will appreciate that generally the accuracy of the analysis as being representative of the tissue section will increase as the area of tissue used for the analysis also increases.

TLR7 (toll like receptor 7) is located on chromosome X and the protein encoded by this gene is a member of the Toll-like receptor (TLR) family which plays a fundamental role in pathogen recognition and activation of innate immunity. The inventors have surprisingly found that this gene is significantly overexpressed in TAMs found in breast cancer, compared to the expression in resident macrophages not associated with breast cancer. Therefore in methods of the invention in which the expression levels of TLR7 are analysed, it is preferred that significant up-regulation of the expression level of TLR7 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, 3.4, or 3.5 in a sample compared to one or more reference values may be indicative of the subject having a particular clinical indication, preferably a diagnosis that breast cancer is present. The skilled person will appreciate that the relative expression levels of TLR7 will depend on the reference values used in the comparison; however, in preferred methods the reference values will correspond to the levels of the biomarkers in resident macrophages from subjects not having cancer, and a log₂ fold change of at least 2, preferably at least 2.5, 3.0, or 3.25, in expression levels of TLR7 will be indicative of a diagnosis that breast cancer is present.

TNFAIP3 (TNF alpha induced protein 3) is located on chromosome 6. The inventors have surprisingly found that this gene is significantly overexpressed in TAMs found in breast cancer, compared to the expression in resident macrophages not associated with breast cancer. Therefore in methods of the invention in which the expression levels of TNFAIP3 are analysed, it is preferred that significant up-regulation of the expression level of TNFAIP3 in a sample from a subject is associated with the subject having a particular clinical indication, preferably a diagnosis that breast cancer is present. For example, a log₂ fold change of at least 0.6, for example a log₂ fold change of at least 0.8, 0.9, 1, 1.1, 1.2, 1.3, 1.4 or 1.5 in a sample compared to one or more reference values may be indicative of the subject having a particular clinical indication, preferably a diagnosis that breast cancer is present. The skilled person will appreciate that the relative expression levels of TNFAIP3 will depend on the reference values used in the comparison; however, in preferred methods the reference values will correspond to the levels of the biomarkers in resident macrophages from subjects not having cancer, and a log₂ fold change of at least 1, preferably at least 1.25, in expression levels of TNFAIP3 will be indicative of a diagnosis that breast cancer is present.

VSIG4 (V-set and immunoglobulin domain containing 4) is located on chromosome X and it encodes a v-set and immunoglobulin-domain. The inventors have surprisingly found that this gene is significantly overexpressed in TAMs found in breast cancer, compared to the expression in resident macrophages not associated with breast cancer. Therefore in methods of the invention in which the expression levels of VSIG4 are analysed, it is preferred that significant up-regulation of the expression level of VSIG4 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of VSIG4 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of VSIG4 will be indicative of a diagnosis that breast cancer is present.

GBP5 (guanylate binding protein 5) is located on chromosome 1. The inventors have surprisingly found that this gene is significantly overexpressed in TAMs found in breast cancer, compared to the expression in resident macrophages not associated with breast cancer. Therefore in methods of the invention in which the expression levels of GBP5 are analysed, it is preferred that significant up-regulation of the expression level of GBP5 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of GBP5 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of GBP5 will be indicative of a diagnosis that breast cancer is present.

HCST (hematopietic cell signal transducer) encodes a transmembrane signaling adaptor that contains a YxxM motif in its cytoplasmic domain. The encoded protein may form part of the immune recognition receptor complex with the C-type lectin-like receptor NKG2D. The inventors have surprisingly found that this gene is significantly overexpressed in TAMs found in breast cancer, compared to the expression in resident macrophages not associated with breast cancer. Therefore in methods of the invention in which the expression levels of HCST are analysed, it is preferred that significant up-regulation of the expression level of HCST in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of HCST will depend on the reference values used in the comparison; however, in preferred methods the reference values will correspond to the levels of the biomarkers in resident macrophages from subjects not having cancer, and a log 2 fold change of at least 1, preferably at least 2, preferably at least 2.5, 3.0, or 3.25 in expression levels of HCST will be indicative of a diagnosis that breast cancer is present.

LILRB4 (Leukocyte immunoglobulin-like receptor subfamily B member 4) is located on chromosome 19. The inventors have surprisingly found that this gene is significantly overexpressed in TAMs found in breast cancer, compared to the expression in resident macrophages not associated with breast cancer. Therefore in methods of the invention in which the expression levels of LILRB4 are analysed, it is preferred that significant up-regulation of the expression level of LILRB4 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of LILRB4 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of LILRB4 will be indicative of a diagnosis that breast cancer is present.

AIF1 (Allograft inflammatory factor 1) is located on chromosome 6. The inventors have surprisingly found that this gene is significantly overexpressed in TAMs found in breast cancer, compared to the expression in resident macrophages not associated with breast cancer. Therefore in methods of the invention in which the expression levels of AIF1 are analysed, it is preferred that significant up-regulation of the expression level of AIF1 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of AIF1 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of AIF1 will be indicative of a diagnosis that breast cancer is present.

PSMB9 (Proteasome subunit beta type-9) is found on chromosome 6. The inventors have surprisingly found that this gene is significantly overexpressed in TAMs found in breast cancer, compared to the expression in resident macrophages not associated with breast cancer. Therefore in methods of the invention in which the expression levels of PSMB9 are analysed, it is preferred that significant up-regulation of the expression level of PSMB9 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of PSMB9 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of PSMB9 will be indicative of a diagnosis that breast cancer is present.

GBP4 (Interferon-induced guanylate-binding protein 4) is a gene related to the superfamily of large GTPases which can be induced mainly by interferon gamma. The inventors have surprisingly found that this gene is significantly overexpressed in TAMs found in breast cancer, compared to the expression in resident macrophages not associated with breast cancer. Therefore in methods of the invention in which the expression levels of GBP4 are analysed, it is preferred that significant up-regulation of the expression level of GBP4 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of GBP4 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of GBP4 will be indicative of a diagnosis that breast cancer is present.

GBP1 (Interferon-induced guanylate-binding protein 1) is a gene related to the superfamily of large GTPases which can be induced mainly by interferon gamma. The inventors have surprisingly found that this gene is significantly overexpressed in TAMs found in breast cancer, compared to the expression in resident macrophages not associated with breast cancer. Therefore in methods of the invention in which the expression levels of GBP1 are analysed, it is preferred that significant up-regulation of the expression level of GBP1 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of GBP1 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of GBP1 will be indicative of a diagnosis that breast cancer is present.

HLA-DOA (HLA class II histocompatibility antigen, DO alpha chain) is found on chromosome 6. The inventors have surprisingly found that this gene is significantly overexpressed in TAMs found in breast cancer, compared to the expression in resident macrophages not associated with breast cancer. Therefore in methods of the invention in which the expression levels of HLA-DOA are analysed, it is preferred that significant up-regulation of the expression level of HLA-DOA in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of HLA-DOA will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of HLA-DOA will be indicative of a diagnosis that breast cancer is present.

CCL3 (Chemokine (C-C motif) ligand 3) is found on chromosome 17. The inventors have surprisingly found that this gene is significantly overexpressed in TAMs found in breast cancer, compared to the expression in resident macrophages not associated with breast cancer. Therefore in methods of the invention in which the expression levels of CCL3 are analysed, it is preferred that significant up-regulation of the expression level of CCL3 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of CCL3 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of CCL3 will be indicative of a diagnosis that breast cancer is present.

CCL4 (Chemokine (C-C motif) ligand 4) is found on chromosome 17. The inventors have surprisingly found that this gene is significantly overexpressed in TAMs found in breast cancer, compared to the expression in resident macrophages not associated with breast cancer. Therefore in methods of the invention in which the expression levels of CCL4 are analysed, it is preferred that significant up-regulation of the expression level of CCL4 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of CCL4 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of CCL4 will be indicative of a diagnosis that breast cancer is present.

CCL8 (Chemokine (C-C motif) ligand 8) is found on chromosome 17. The inventors have surprisingly found that this gene is significantly overexpressed in TAMs found in breast cancer, compared to the expression in resident macrophages not associated with breast cancer. Therefore in methods of the invention in which the expression levels of CCL8 are analysed, it is preferred that significant up-regulation of the expression level of CCL8 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of CCL8 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of CCL8 will be indicative of a diagnosis that breast cancer is present.

In the experiments described below, the inventors show that CCL8 expression at the RNA level is increased in TAMs, particularly TAMs associated with a poor prognosis; CCL8 is produced in these TAMs and then secreted into the surrounding tissues. This has been shown by the inventors at both the RNA and the protein level. Therefore the skilled person will appreciate that CCL8 expression can be analyzed in the methods of the invention by looking at either nucleic acid or protein levels. The skilled person will also appreciate, and it is illustrated below, that when nucleic acid levels of CCL8 are analyzed, for example using RNA FISH or intracellular FACS, it is preferable to analyze the expression in samples enriched for macrophages, and/or to specifically analyze the expression in the macrophages of the sample as explained above, whilst when protein levels of CCL8 are analyzed this is less important so that it is preferable to analyze samples, for example histological sections, of whole tissue. Secreted CCL8 protein can be detected in media using an enzyme-linked immunosorbent assay (ELISA).

NCF1C (Putative neutrophil cytosol factor 1C) is found on chromosome 7. The inventors have surprisingly found that this gene is significantly overexpressed in TAMs found in breast cancer, compared to the expression in resident macrophages not associated with breast cancer. Therefore in methods of the invention in which the expression levels of NCF1C are analysed, it is preferred that significant up-regulation of the expression level of NCF1C in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of NCF1C will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of NCF1C will be indicative of a diagnosis that breast cancer is present.

LAP3 (Leucine Aminopeptidase 3) is found on chromosome 4. The inventors have surprisingly found that this gene is significantly overexpressed in TAMs found in breast cancer, compared to the expression in resident macrophages not associated with breast cancer. Therefore in methods of the invention in which the expression levels of LAP3 are analysed, it is preferred that significant up-regulation of the expression level of LAP3 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of LAP3 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of LAP3 will be indicative of a diagnosis that breast cancer is present.

LAIR1 (Leukocyte-associated immunoglobulin-like receptor 1) is found on chromosome 19. The inventors have surprisingly found that this gene is significantly overexpressed in TAMs found in breast cancer, compared to the expression in resident macrophages not associated with breast cancer. Therefore in methods of the invention in which the expression levels of LAIR1 are analysed, it is preferred that significant up-regulation of the expression level of LAIR1 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of LAIR1 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of LAIR1 will be indicative of a diagnosis that breast cancer is present.

FOLR2 (Folate receptor beta) is found on chromosome 11. The inventors have surprisingly found that this gene is significantly overexpressed in TAMs found in breast cancer, compared to the expression in resident macrophages not associated with breast cancer. Therefore in methods of the invention in which the expression levels of FOLR2 are analysed, it is preferred that significant up-regulation of the expression level of FOLR2 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of FOLR2 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of FOLR2 will be indicative of a diagnosis that breast cancer is present.

TCN2 (transcobalamin 2) is found on chromosome 22. The inventors have surprisingly found that this gene is significantly overexpressed in TAMs found in breast cancer, compared to the expression in resident macrophages not associated with breast cancer. Therefore in methods of the invention in which the expression levels of TCN2 are analysed, it is preferred that significant up-regulation of the expression level of TCN2 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of TCN2 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of TCN2 will be indicative of a diagnosis that breast cancer is present.

PLTP (Phospholipid transfer protein) is found on chromosome 20. The inventors have surprisingly found that this gene is significantly overexpressed in TAMs found in breast cancer, compared to the expression in resident macrophages not associated with breast cancer. Therefore in methods of the invention in which the expression levels of PLTP are analysed, it is preferred that significant up-regulation of the expression level of PLTP in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of PLTP will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of PLTP will be indicative of a diagnosis that breast cancer is present.

DOK2 (Docking protein 2) is found on chromosome 8. The inventors have surprisingly found that this gene is significantly overexpressed in TAMs found in breast cancer, compared to the expression in resident macrophages not associated with breast cancer. Therefore in methods of the invention in which the expression levels of DOK2 are analysed, it is preferred that significant up-regulation of the expression level of DOK2 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of DOK2 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of DOK2 will be indicative of a diagnosis that breast cancer is present.

GIMAP6 (GTPase IMAP family member 6) is found on chromosome 7. The inventors have surprisingly found that this gene is significantly overexpressed in TAMs found in breast cancer, compared to the expression in resident macrophages not associated with breast cancer. Therefore in methods of the invention in which the expression levels of GIMAP6 are analysed, it is preferred that significant up-regulation of the expression level of GIMAP6 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of GIMAP6 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of GIMAP6 will be indicative of a diagnosis that breast cancer is present.

CD40 (cluster of differentiation 40) is mainly found on antigen presenting cells, chromosome 20. The inventors have surprisingly found that this gene is significantly overexpressed in TAMs found in breast cancer, compared to the expression in resident macrophages not associated with breast cancer. Therefore in methods of the invention in which the expression levels of CD40 are analysed, it is preferred that significant up-regulation of the expression level of CD40 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of CD40 will depend on the reference values used in the comparison; however, in preferred methods the reference values will correspond to the levels of the biomarkers in resident macrophages from subjects not having cancer, and a log 2 fold change of at least 1, preferably at least 2, preferably at least 2.5, 3.0, or 3.25 in expression levels of CD40 will be indicative of a diagnosis that breast cancer is present.

FCN1 (Ficolin-1) is found on chromosome 9. The inventors have surprisingly found that this gene is significantly overexpressed in TAMs found in breast cancer, compared to the expression in resident macrophages not associated with breast cancer. Therefore in methods of the invention in which the expression levels of FCN1 are analysed, it is preferred that significant up-regulation of the expression level of FCN1 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of FCN1 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of FCN1 will be indicative of a diagnosis that breast cancer is present.

CD4 (cluster of differentiation 4) is found on chromosome 12. The inventors have surprisingly found that this gene is significantly overexpressed in TAMs found in breast cancer, compared to the expression in resident macrophages not associated with breast cancer. Therefore in methods of the invention in which the expression levels of CD4 are analysed, it is preferred that significant up-regulation of the expression level of CD4 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of CD4 will depend on the reference values used in the comparison; however, in preferred methods the reference values will correspond to the levels of the biomarkers in resident macrophages from subjects not having cancer, and a log 2 fold change of at least 1, preferably at least 2, preferably at least 2.5, 3.0, or 3.25 in expression levels of CD4 will be indicative of a diagnosis that breast cancer is present.

VAV1 (Proto-oncogene vav) is found on chromosome 19. The inventors have surprisingly found that this gene is significantly overexpressed in TAMs found in breast cancer, compared to the expression in resident macrophages not associated with breast cancer. Therefore in methods of the invention in which the expression levels of VAV1 are analysed, it is preferred that significant up-regulation of the expression level of VAV1 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of VAV1 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of VA VI will be indicative of a diagnosis that breast cancer is present.

FGD2 (FYVE, RhoGEF and PH domain-containing protein 2) is found on chromosome 6. The inventors have surprisingly found that this gene is significantly overexpressed in TAMs found in breast cancer, compared to the expression in resident macrophages not associated with breast cancer. Therefore in methods of the invention in which the expression levels of FGD2 are analysed, it is preferred that significant up-regulation of the expression level of FGD2 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of FGD2 will depend on the reference values used in the comparison; however, in preferred methods the reference values will correspond to the levels of the biomarkers in resident macrophages from subjects not having cancer, and a log 2 fold change of at least 1, preferably at least 2, preferably at least 2.5, 3.0, or 3.25 in expression levels of FGD2 will be indicative of a diagnosis that breast cancer is present.

LST1 (Leukocyte-specific transcript 1 protein) is found on chromosome 6. The inventors have surprisingly found that this gene is significantly overexpressed in TAMs found in breast cancer, compared to the expression in resident macrophages not associated with breast cancer. Therefore in methods of the invention in which the expression levels of LST1 are analysed, it is preferred that significant up-regulation of the expression level of LST1 in a sample from a subject is associated with the subject having a particular clinical indication, preferably a diagnosis that breast cancer is present. For example, a log₂ fold change of at least 1, for example a log₂ fold change of at least 1.5, 2.0, 2.5, 2.6, 2.7, 2.8, 2.9, 3.0, 3.1, 3.2, 3.3, or 3.4, in a sample compared to one or more reference values may be indicative of the subject having a particular clinical indication, preferably a diagnosis that breast cancer is present. The skilled person will appreciate that the relative expression levels of LST1 will depend on the reference values used in the comparison; however, in preferred methods the reference values will correspond to the levels of the biomarkers in resident macrophages from subjects not having cancer, and a log₂ fold change of at least 1, preferably at least 2, preferably at least 2.5, 3.0, or 3.25 in expression levels of LST1 will be indicative of a diagnosis that breast cancer is present.

The biological samples are analysed to determine the expression levels of the biomarkers. “Gene expression”, or more simply “expression” is the process by which information from a gene is used in the synthesis of a functional gene product, such as a protein or non-coding RNA (ncRNA). As used herein, the term “expression” includes RNA (for example mRNA) transcription. Thus suitably the expression level for a biomarker may be determined by looking at the amount of a target molecule selected from the group consisting of the protein expressed from the biomarker and a polynucleotide molecule encoding the biomarker, or a nucleic acid complementary thereto. It is preferred that the target molecule is a nucleic acid molecule, and highly preferred that it is an RNA molecule, for example mRNA, transcribed from the biomarker or a cDNA molecule complementary thereto.

The levels of the target molecules, which are representative of expression of the biomarkers in the biological sample, may be investigated for example using specific binding partners, polymerase chain reaction (PCR) and/or sequencing techniques. The binding partners may be selected from the group consisting of complementary nucleic acids, aptamers, and antibodies or antibody fragments. Preferably the levels of the biomarkers in the biological sample are investigated using a nucleic acid probe having a sequence which is complementary to the sequence of the relevant mRNA, ncRNA or cDNA against which it is targeted.

Suitable classes of binding partners for any given biomarker will be apparent to the skilled person, and are discussed further below. The expression levels of the biomarkers in the biological sample may be detected by direct assessment of binding between the target molecules and binding partners. The levels of the biomarkers in the biological sample may be detected using a reporter moiety attached to a binding partner. Preferably the reporter moiety is selected from the group consisting of fluorophores; chromogenic substrates; and chromogenic enzymes.

### Methods of Diagnosis, Prognosis and Treatment

As explained in the Experimental Results section, the methods of the invention are able to distinguish at least between samples from individuals with and without breast cancer, stratify the breast cancer patients according to ER status, tumor grade and survival rate, and/or predict outcome. Therefore the term “clinical indication” should be interpreted broadly to refer to clinical details that may generally be associated with a particular breast tissue sample known as comprising or potentially comprising breast cancer. In particular “a clinical indication” may refer to the presence or absence of breast cancer generally, the presence or absence of a particular type of breast cancer, the particular grade of breast cancer (e.g. grade I, II, or III, or combinations thereof), the status with respect to the presence or absence of one or more receptors, (e.g. ER+, ER−, HER2+, HER2−, PR+, PR−, and combinations thereof), a good response to treatment, a poor response to treatment, a poor survival rate, a good survival rate, a good prognosis/outcome, an intermediate prognosis/outcome, a poor prognosis/outcome, the presence of local and/or distant metastasis, the absence of local and/or distant metastasis, and/or recurrence of the breast cancer following treatment or no recurrence of the breast cancer following treatment.

Therefore in some methods of the invention, the clinical indication is of whether cancer is present or not in the breast tissue of the subject. This diagnosis may be made, for example, by using reference values corresponding to, or having a defined relationship with, macrophage biomarker expression in breast tissue samples from patients not having breast cancer; the differential expression for a clinical indication of cancer being present when such reference values are used is indicated above, see for example Tables 1 and 2. Alternatively, the diagnosis may be made, for example, by using reference values corresponding to, or having a defined relationship with, TAM biomarker expression in breast tissue samples from patients having breast cancer, such that a clinical indication of cancer being present could be made based on the lack of significant differential expression when such reference values are used. Furthermore, the diagnosis could be made, for example, by using one or more gene expression signatures as reference values, wherein gene expression signatures are used that are representative of macrophage biomarker expression in breast tissue samples from patients not having breast cancer and/or TAM biomarker expression in breast tissue samples from patients having breast cancer; comparison of the biomarker expression in the biological sample with the gene expression signatures can then be carried out to determine with which signature the biological sample has most similarity (correlates), such that the subject identifies with the patients of that gene expression signature from a cancer diagnosis perspective.

In some methods of the invention the clinical indication may be of a particular grade of breast cancer, such as invasive breast cancer grade I, grade II, or grade Ill, or a combination thereof such as “low grade” (grade I-II) or “high grade” (grade Ill), or DCIS low, intermediate or high grade. The determination of breast cancer grade could be made, for example, by using gene expression signatures as reference values, wherein gene expression signatures are used that are selected from those representative of macrophage biomarker expression in breast tissue samples from patients not having breast cancer and/or TAM biomarker expression in breast tissue samples from patients having a particular grade or combination of grades of invasive breast cancer and/or TAM biomarker expression in breast tissue samples from patients having a particular grade or DCIS; comparison of the biomarker expression in the biological sample with the gene expression signatures can then be carried out to determine with which signature the biological sample has most similarity (correlates), such that the subject identifies with the patients of that gene expression signature from a cancer grade perspective.

In some methods of the invention the clinical indication may be of a particular outcome for the breast cancer, such as good outcome, intermediate outcome, or poor outcome. The determination of likely outcome could be made, for example, by using gene expression signatures as reference values, wherein gene expression signatures are used that are selected from those representative of TAM biomarker expression in breast tissue samples from patients having a particular outcome; comparison of the biomarker expression in the biological sample with the gene expression signatures can then be carried out to determine with which signature the biological sample has most similarity (correlates), such that the subject identifies with the patients of that gene expression signature from a cancer outcome perspective. Allocation of patients to outcome groups, in order to generate the gene expression signatures, may be based, for example, on patient response to treatment, presence, absence or extent of any metastases, and/or survival rate of the patient group, as explained further below.

In some methods of the invention the clinical indication may be of the status with respect to the presence or absence of one or more receptors, such as the ER, the HER2 receptor and/or the progesterone receptor (PR). Therefore the clinical indication may be ER+, ER−, HER2+, HER2−, PR+, PR−, ER−/HER2−, HER2−/PR−, ER+/HER2−, HER2+/PR−, ER−/HER2−/PR−, ER+/HER2+/PR+, ER+/HER2−/PR−, ER+/HER2+/PR−, ER−/HER2−/PR+, ER−/HER2+/PR−, and/or some other possible combination. The determination of receptor status could be made, for example, by using gene expression signatures as reference values, wherein gene expression signatures are used that are selected from those representative of TAM biomarker expression in breast tissue samples from patients having a particular known receptor status or combination of known receptor statuses; comparison of the biomarker expression in the biological sample with the gene expression signatures can then be carried out to determine with which signature the biological sample has most similarity (correlates), such that the subject identifies with the patients of that gene expression signature from a receptor status perspective.

In some methods, biomarker expression may be used to independently stratify breast cancer patients, to provide additional information above and beyond the current classifications, such as “ER status”, “Basal type”, etc, and thereby, for example, provide information regarding most likely effective therapy for patients, particularly immunotherapy. In this way, the tumor microenvironment may provide completely new and useful subsets of classifications.

The term “diagnosing” or “diagnosis” as used herein in the context of breast cancer should be taken as allowing a distinction to be made regarding a breast tissue sample; the term is used to mean an indication of the presence or absence of breast cancer and/or an indication of particulars of the disease, for example receptor status or tumor grade.

The term “prognosis” as used herein refers to the likelihood of the clinical outcome for a subject having breast cancer, and is a representation of the likelihood (probability) that the subject will survive (such as for one, two, three, four or five years) and/or the likelihood (probability) that the tumor will progress in grade and/or metastasize. The term “prediction” is used herein to refer to the likelihood that a patient will respond either favourably or unfavourably to a therapy, drug or set of drugs, and also the extent of those responses. The prognostic and predictive methods of the invention can be used clinically to make treatment decisions by choosing the most appropriate treatment modalities for any particular patient. The methods are thus valuable tools in predicting if a patient is likely to respond favourably to a treatment regimen, such as surgical intervention, chemotherapy with a given drug or drug combination, and/or radiation therapy, as explained further below. Methods of prognosis or prediction may involve, for example, using gene expression signatures as reference values, wherein gene expression signatures are used that are representative TAM biomarker expression in breast tissue samples from patients having a particular known outcome; comparison of the biomarker expression in the biological sample with the gene expression signatures can then be carried out to determine with which signature the biological sample has most similarity (correlates), such that the subject identifies with the patients of that gene expression signature from a prognosis and/or prediction perspective.

Other physical or biological measurements may be taken, or tests carried out, in conjunction with the measurement of biomarker expression levels as part of the methods of the invention. Preferably the methods of the invention, or at least preferably those that do not involve treatment of the subject, are performed in vitro and/or ex vivo and/or are not practised on the subject's body. For the avoidance of doubt, it should be noted that the present invention can be used for both initial diagnosis of cancer and for ongoing monitoring of cancer, e.g. indicating the continued presence of cancer despite treatment (response to, or outcome following, treatment) or indicating the presence of cancer after a period of being “cancer free” following treatment (assessing recurrence).

Where methods are described herein as indicating a “poor” diagnosis, prognosis, or outcome, this is used to mean that the breast cancer is clinically associated with more developed, advanced, aggressive and/or extensive disease and so a poor clinical outcome. Clinical outcome refers to the health status of a patient following treatment for a disease or disorder, or in the absence of treatment, and so clinical outcomes include, but are not limited to, an increase in the length of time until death, a decrease in the length of time until death, an increase in the chance of survival, an increase in the risk of death, survival, disease-free survival, chronic disease, metastasis, advanced or aggressive disease, disease recurrence, death, and favorable or poor response to therapy. For example, a method indicating a poor clinical diagnosis, prognosis, or outcome may indicate a higher grade of breast cancer, the presence of receptors such as for oestrogen, the protein HER2 and/or progesterone, a lower chance of response to treatment, a greater chance of recurrence following treatment, and/or a reduced life expectancy. In comparison, a “good” diagnosis, prognosis, or outcome, is used to mean that the breast cancer is clinically associated with less developed, advanced, aggressive and/or extensive disease and so a good clinical outcome. For example, it may indicate a lower grade of breast cancer, the absence of receptors such as for oestrogen, the protein HER2 and/or progesterone, a higher chance of response to treatment, a lower chance of recurrence following treatment, and/or minimal impact of the breast cancer on life expectancy.

The methods of the invention may be used to provide a clinical indication, for example diagnose cancer, in a subject showing symptoms consistent with such disease. Alternatively, the methods of the invention may be used to diagnose cancer in a subject that appears asymptomatic. Cancer may be asymptomatic, for example, during the early stages of recurrence of the disease.

As used herein the term “cancer” includes: cancer generically; groups or sub-groups of cancers originating from specific organs, tissues and/or cell types; cancer originating from a specific organ, tissue and/or cell type; and cancers of unknown primary origin. The invention relates to cancers found in breast tissue. Breast cancer can be detected and/or indicated in the methods of the invention. Breast cancer includes, for example, ductal carcinoma in situ (DCIS), invasive ductal carcinoma, invasive lobular carcinoma, or inflammatory breast cancer. Preferably the cancer will be an invasive breast cancer. The breast cancer may be primary or metastatic, and may be a recurrent breast cancer. The methods may suitably comprise comparing the results obtained with the results obtained in an equivalent (typically identical) procedure carried out previously on a biological sample of breast tissue from the same subject.

The methods of treatment may involve any of the treatments known in the art for the cancer diagnosed, for example one or more treatments selected from the group consisting of surgery, radiation therapy, chemotherapy, immunotherapy, hormone therapy, and targeted therapy. The therapy may, for example, be used to remove the entire tumor, to debulk the tumor, and/or to ease the cancer symptoms.

Surgery involves removing or destroying tumor tissue and may be open or minimally invasive. It may include, for example, the use of sharp tools to cut the body, cryosurgery, lasers, hyperthermia and/or photodynamic therapy.

Radiation therapy involves the use of high doses of radiation to kill cancer cells and shrink tumors. Treatment using radiation therapy in accordance with the invention includes the use of external beam radiation therapy, where an external source is used to aim radiation at the affected part(s) of the body, and internal radiation therapy (brachytherapy), where a solid or liquid radiation source is put into the body. Radiation therapies of use in embodiments of the invention include the use of external x-rays or gamma rays, interstitial brachytherapy, intracavitary brachytherapy, samarium-153-lexidronam (Quadramet) and strontium-89 chloride (Metastron).

Chemotherapy involves the use of chemicals that target the fast dividing cancer cells. It may be used on its own or in combination with other cancer therapies. Chemotherapy drugs of use in embodiments of the invention include one or more of Abraxane (Abraxane), Bendamustine, (Levact), Bleomycin, Capecitabine (Xeloda), Carboplatin, Carmustine (BiCNU), Chlorambucil (Leukeran), Cisplatin, Cyclophosphamide (Cytoxan), Cytarabine, Dacarbazine (DTIC), Dactinomycin (Cosmegen Lyovac), Daunorubicin, Docetaxel (Taxotere), Doxorubicin (Adriamycin), Epirubicin (Pharmorubicin), Eribulin (Halaven), Etoposide (VP-16, Etopophos, Vepesid), Fluorouracil (5FU), Gemcitabine (Gemzar), Idarubicin (Zavedos), Ifosfamide (Mitoxana), Irinotecan (Campto), Liposomal doxorubicin (DaunoXome), Lobaplatin, Melphalan (Alkeran), Methotrexate (Maxtrex), Mitomycin (Mitomycin C), Mitoxantrone, Oxaliplatin (Eloxatin), Paclitaxel (Taxol), Pemetrexed (Alimta), Tegafur (Utefos), Temozolomide (Temodal), Thiotepa, Topotecan (Hycamtin), Trabectedin (Yondelis), Venetoclax (Venclexta), Vinblastine (Velbe), Vincristine (Oncovin), Vindesine (Eldisine), and Vinorelbine (Navelbine).

Immunotherapy includes treatment that help the subject's immune system to target the cancer cells. Immunotherapies of use in embodiments of the invention include monoclonal antibodies such as those targeting CTLA4 or PD1 or PDL1, adoptive cell transfer which boosts the ability of T cells to fight the cancer, cytokines such as interferons and interleukins, vaccines, immune system stimulators (CpG, Iquimod, 852A etc) such as engagement of Toll like receptors and live tumor targeted viruses or bacteria.

Hormone therapy blocks the body's ability to produce hormones, or interferes with how the hormones behave. Hormone therapies of use in embodiments of the invention include anti-estrogens e.g Raloxifene hydrochloride (Evista), medroxyprogesterone, Dromostanolone propionate (Masteril), luteinising hormone blockers e.g Goserelin (Zoladex), gonadotropin-releasing hormone (GnRH) analogues e.g Leuprolide acetate (Lucrin), Triptorelin pamoate (Decapeptyl SR), Buserelin acetate, and aromatase inhibitors e.g. formestane (Lentaron).

Targeted therapy involves selecting drugs that specifically target changes that have occurred during the development of the specific cancer in the subject's body. Examples of targeted therapies that may be used in embodiments of the invention include small-molecule drugs and monoclonal antibodies. Preferably the targeted therapies in the embodiments of the invention will include one or more from the group consisting of Trastuzumab (Herceptin), ramucirumab (Cyramza), Vismodegib (Erivedge), sonidegib (Odomzo), Atezolizumab (Tecentriq), nivolumab (Opdivo), Bevacizumab (Avastin), Everolimus (Afinitor), tamoxifen (Nolvadex), afimoxifene, toremifene (Fareston), fulvestrant (Faslodex), anastrozole (Arimidex), exemestane (Aromasin), lapatinib (Tykerb), letrozole (Femara), pertuzumab (Perjeta), ado-trastuzumab emtansine (Kadcyla), palbociclib (lbrance), ribociclib (Kisqali), abemaciclib (Verzenio), Alpelisib (BYL-719), Ipatasertib (RG-7440/GDC-0068), plinabulin (NPI-2358), tucidinostat (Chidamide), Cetuximab (Erbitux), panitumumab (Vectibix), ramucirumab (Cyramza), pembrolizumab (Keytruda), Denosumab (Xgeva), Margetuximab, Mogamulizumab (Poteligeo), sorafenib (Nexavar), pazopanib (Votrient), temsirolimus (Torisel), axitinib (Inlyta), cabozantinib (Cabometyx), lenvatinib mesylate (Lenvima), crizotinib (Xalkori), erlotinib (Tarceva), gefitinib (Iressa), afatinib dimaleate (Gilotrif), Ipilimumab (Yervoy), vemurafenib (Zelboraf), trametinib (Mekinist), neratinib (Nerlynx), pyrotinib (SHR-1258/HTI-1001), cobimetinib (Cotellic), Bortezomib (Velcade), panobinostat (Farydak), daratumumab (Darzalex), ixazomib citrate (Ninlaro), olaparib (Lynparza), rucaparib camsylate (Rubraca), niraparib (Zejula), talazoparib (Talzenna), enzalutamide (Xtandi), abiraterone acetate (Zytiga), Cabozantinib (Cometriq), and vandetanib (Caprelsa).

In some embodiments of the methods of treatment provided herein, the breast cancer treatment is one or more selected from the group consisting of surgery, radiation therapy, chemotherapy, hormonal therapy, immunotherapy, and targeted therapy. Preferably the chemotherapy involves treatment with one or more drugs selected from the group consisting of Capecitabine (Xeloda), Carboplatin (Paraplatin), Cisplatin (Platinol), Cyclophosphamide (Neosar), Docetaxel (Docefrez, Taxotere), Doxorubicin (Adriamycin), Pegylated liposomal doxorubicin (Doxil), Epirubicin (Ellence), Fluorouracil (5-FU, Adrucil), Gemcitabine (Gemzar), Methotrexate (multiple brand names), Paclitaxel (Taxol), Protein-bound paclitaxel (Abraxane), Vinorelbine (Navelbine), Eribulin (Halaven), mitoxantrone (Mitozantrone or Onkotrone), mitomycin C, Ixabepilone (Ixempra) and megestrol (Megace). Preferably the hormonal therapy involves treatment with one or more treatments selected from the group consisting of Tamoxifen, aromatase inhibitors (Als) such as Anastrozole (Arimidex) and Exemestane (Aromasin), Letrozole (Femara), Fulvestrant (Faslodex), ovarian suppression or ablation such as using goserelin (Zoladex), megestrol acetate (Megace) and high-dose estradiol. Preferably the targeted therapy and/or immunotherapy involves treatment with one or more selected from the group consisting of palbociclib (lbrance), Everolimus (Afinitor), Trastuzumab, Pertuzumab (Perjeta), Ado-trastuzumab emtansine or T-DM1 (Kadcyla), Lapatinib (Tykerb), Bisphosphonates, and Denosumab (Xgeva).

The biomarkers of the invention allow a diagnosis, prognosis and/or prediction to be made regarding the breast cancer. Therefore in some embodiments of the methods of the invention, particularly embodiments of the methods of treating breast cancer, this knowledge will be used to decide the best treatment or minimal treatment option. For example, if the expression of the biomarkers in the sample indicate that the breast cancer is of a high grade or more likely to recur, and/or that the subject providing the sample is likely to have a reduced life expectancy, then a more aggressive treatment schedule may be used. For example, systemic therapy may be used before and/or after surgery, and/or treatments may comprise immunomodulating therapies, such as anti-TAM therapy (eg anti-CSF1R) or other macrophage reprogramming therapies, or anti-PD1, -PDL1 or CTLA4 or NK cell therapy, after surgery or chemotherapy, and/or treatments may be given for longer or at higher concentrations. Similarly, if the expression of the biomarkers in the sample indicate a good outcome for breast cancer treatment, then the course of treatment followed may be less aggressive, for example it may be decided that there is no need to use adjuvant chemotherapy after surgery and/or any treatments may be given for less time or at lower concentrations, compared to treatments that would be given for a more aggressive breast cancer. Knowledge of the receptor status of the breast cancer may further determine the most appropriate course of treatment. Such as to determine the type of immunotherapy eg anti-PD1, anti CTLA4 or combination thereof and/or the use of other therapies such as NKtherapy or alternative T-Cell checkpoint inhibitor therapies.

### Binding Partners

In certain embodiments of the invention, expression levels of the biomarkers in a biological sample may be investigated using binding partners which bind or hybridize specifically to a target molecule for the biomarkers, or a fragment thereof. In relation to the present invention the term ‘binding partners’ may include any ligands, which are capable of binding specifically to the relevant biomarker and/or nucleotide or peptide variants thereof with high affinity. Said ligands include, but are not limited to nucleic acids (DNA or RNA), proteins, peptides, antibodies, synthetic affinity probes, carbohydrates, lipids, artificial molecules or small organic molecules such as drugs. In certain embodiments the binding partners may be selected from the group comprising: complementary nucleic acids; aptamers; antibodies or antibody fragments. In the case of detecting mRNAs and cDNAs, nucleic acids represent highly suitable binding partners.

In the context of the present invention, a binding partner specific to a biomarker should be taken as requiring that the binding partner should be capable of binding to at least one target molecule for such biomarker in a manner that can be distinguished from non-specific binding to molecules that are not target molecules for biomarkers. A suitable distinction may, for example, be based on distinguishable differences in the magnitude of such binding.

In preferred embodiments of the methods or devices of the invention, the target molecule for the biomarker is a nucleic acid, preferably an mRNA or cDNA molecule, and the binding partner is selected from the group consisting of complementary nucleic acids and aptamers.

Suitably the binding partner is a nucleic acid molecule (typically DNA, but it can be RNA) having a sequence which is complementary to the sequence of the relevant mRNA, ncRNA or cDNA against which is targeted. Such a nucleic acid is often referred to as a ‘probe’ (or a reporter or an oligo) and the complementary sequence to which it binds is often referred to as the ‘target’. Probe-target hybridization is usually detected and quantified by detection of fluorophore-, silver-, or chemiluminescence-labeled targets to determine relative abundance of nucleic acid sequences in the target.

Probes can be from 25 to 1000 nucleotides in length. However, lengths of 30 to 100 nucleotides are preferred, and probes of around 50 nucleotides in length are commonly used with great success in complete transcriptome analysis.

While the determination of suitable probes can be difficult, e.g. in very complex arrays, there are many commercial sources of complete transcriptome arrays available, and it is routine to develop bespoke arrays to detect any given set of specific mRNAs using publicly available sequence information. Commercial sources of microarrays for transciptome analysis include Illumina and Affymetrix.

TABLE 3
Probe sequences and accession numbers for the biomarkers of Table 1.

| HGNC | | | SEQ ID | |
|---|---|---|---|---|
| Symbol | Probe Sequence (Illumina Human HT 12 V4 probes) | Probe Name | NO. | Gene ID |
| C1QA | ACCAACAAGGGGCTCTTCCAGGTGGTGTCAGGGGGCATGGTGCTTCAGCT | ILMN_1737918 | 1 | ENSG00000173372 |
| C1QB | CAGGCCACCGACAAGAACTCACTACTGGGCATGGAGGGTGCCAACAGCAT | ILMN_1796409 | 2 | ENSG00000173369 |
| IRF8 | GCCAGATATGCCTGTTTCCTTTTCCCAGCACCATGCCTGTGGAGGGGACA | ILMN_181786 | 3 | ENSG00000140968 |
| CCL2 | CAGGATTCCATGGACCACCTGGACAAGCAAACCCAAACTCCGAAGACTTG | ILMN_25185 | 4 | ENSG00000108691 |
| GBP5 | GCAGGAACAACAGATGCAGGAACAGGCTGCACAGCTCAGCACAACATTCC | ILMN_24462 | 5 | ENSG00000154451 |
| HCST | GGTGGCACAGGAACCCCCGCCCCAACTTTTGGATTGTAATAAAACAATTG | ILMN_8098 | 6 | ENSG00000126264 |
| LILRB4 | CCTTCTTCCTCCCAGGAAAGGGGACGTTCAGCTGAGCCGAGTGTGTATAC | ILMN_20705 | 7 | ENSG00000186818 |
| AIF1 | GTCTCCCCACCTCTACCAGCATCTGCTGAGCTATGAGCCAAACCAGGGAT | ILMN_17792 | 8 | ENSG00000204472 |
| PSMB9 | ACAAGCTGTCCCCGCTGCACGAGCGCATCTACTGTGCACTCTCTGGTTCA | ILMN_12611 | 9 | ENSG00000240065 |
| GBP4 | CCATGGGCCTTTTCACAGGGGACACAGGCTTCTTAAAACAACCCGGCTTC | ILMN_172048 | 10 | ENSG00000162654 |
| GBP1 | GTTCTCCAGAGGAAGGTGGAAGAAACCATGGGCAGGAGTAGGAATTGAGT | ILMN_28413 | 11 | ENSG00000117228 |
| HLA-DOA | GGCCAAACTTGGAGCAGGTGTCCATCCCAGCCCTGTGTAGTTAGAGCAGG | ILMN_27857 | 12 | ENSG00000204252 |
| CCL4 | AGGTGTCATTTCCATTATTTATATTAGTTTAGCCAAAGGATAAGTGTCCT | ILMN_138034 | 13 | ENSG00000275302 |
| NCF1C | CAGGCTACTTTCCGTCCATGTACCTGCAAAAGTCGGGGCAAGACGTGTCC | ILMN_175406 | 14 | ENSG00000165178 |
| LAP3 | CCAACAAAGATGAAGTTCCCTATCTACGGAAAGGCATGACTGGGAGGCCC | ILMN_12514 | 15 | ENSG00000002549 |
| TNFAIP3 | CCCCAGAGATAAAGGCTGCCATTTTGGGGGTCTGTACTTATGGCCTGAAA | ILMN_2315 | 16 | ENSG00000118503 |
| ITGB2 | GGAGACTTGAGGAGGGCTTGAGGTTGGTGAGGTTAGGTGCGTGTTTCCTG | ILMN_138267 | 17 | ENSG00000160255 |
| LAIR1 | CTCCTGGTCCTCTTCTGCCTCCATCGCCAGAATCAGATAAAGCAGGGGCC | ILMN_38361 | 18 | ENSG00000167613 |
| FOLR2 | GAATGCTGGTGAGATGCTTCATGGGACTGGGGGTCTCCTGCTCAGTCTGG | ILMN_11875 | 19 | ENSG00000165457 |
| CD83 | TGGGTGCTCGCCCACTTGTCCCACTATCTGGGTGCATGATCTTGAGCAAG | ILMN_181889 | 20 | ENSG00000112149 |
| SIGLEC1 | GAGACCACGCAGCTCATTGATCCTGATGCAGCCACATGTGAGACCTCAAC | ILMN_34388 | 21 | ENSG00000088827 |
| TCN2 | CTGCAGGTCTCCCATGAAGGCCACCCCATGGTCTGATGGGCATGAAGCAT | ILMN_6136 | 22 | ENSG00000185339 |
| PLTP | TTGCCAAAGGGCTGCGAGAGGTGATTGAGAAGAACCGGCCTGCTGATGTC | ILMN_20706 | 23 | ENSG00000100979 |
| DOK2 | GAGCCGGCCCTGCATGGAGGAAAATGAATTGTACAGCAGCGCAGTCACAG | ILMN_21820 | 24 | ENSG00000147443 |
| GIMAP6 | GCCATCCCCCATCTTCCCTAGACACAGCAGACATCTGAGAAAGCTTCAGC | ILMN_1865 | 25 | ENSG00000133561 |
| CD40 | CTGGAAGGGTACACAGAAAACCCACAGCTCGAAGAGTGGTGACGTCTGGG | NM_152854.2 | 26 | ENSG00000101017 |
| CCL3 | CCTCTGCACCATGGCTCTCTGCAACCAGTTCTCTGCATCACTTGCTGCTG | ILMN_1999 | 27 | ENSG00000277632 |
| CCL8 | GTCATTGTTCTCCCTCCTACCTGTCTGTAGTGTTGTGGGGTCCTCCCATG | ILMN_15634 | 28 | ENSG00000108700 |
| FCN1 | CCAGCTCAGTCAAGCCGCCACATGCCCACAACCTCACCAGAGGGAGAATT | ILMN_11290 | 29 | ENSG00000085265 |
| CD4 | TGCCCCACACCCTCCCTTACCCTCCTCCAGACCATTCAGGACACAGGGAA | ILMN_28131 | 30 | ENSG00000010610 |
| VAV1 | TCAAGATCCTTAACAAGAAGGGACAGCAAGGCTGGTGGCGAGGGGAGATC | ILMN_20639 | 31 | ENSG00000141968 |
| TLR7 | CAGCGTGCATGTGTTCAAGCCTTAGATTGGCGATGTCGTATTTTCCTCAC | ILMN_3324 | 32 | ENSG00000196664 |
| FGD2 | AGAGAGCAAACTACCACAACCAATGGTTGAGCCCCTGTCAAGTGCCAGTC | ILMN_13097 | 33 | ENSG00000146192 |
| LST1 | TGCTGAGAACAAACCCACCTGAGCACCCCAGACACCTTCCTCAACCCAGG | ILMN_22632 | 34 | ENSG00000204482 |
| VSIG4 | GGCCCTTCTAGTATCTCTGCCGGGGGCTTCTGGTACTCCTCTCTAAATAC | ILMN_18144 | 35 | ENSG00000155659 |
| CLEC7A | GACTCAGAGATTCTCTTTTGTCCACAGACAGTCATCTCAGGAGCAGAAAG | ILMN_33785 | 36 | ENSG00000172243 |
| C1QC | ATGGTGGGCATCCAGGGCTCTGACAGCGTCTTCTCCGGCTTCCTGCTCTT | ILMN_18780 | 37 | ENSG00000159189 |

In one embodiment the probe sequences will comprise sequences selected from those listed in Table 3. However, nucleotide probe sequences may be designed to any sequence region of the biomarker transcripts (accession numbers listed in Table 3) or a variant thereof. Nucleotide probe sequences, for example, may include, but are not limited to those listed in Table 3. The person skilled in the art will appreciate that equally effective probes can be designed to different regions of the transcript than those targeted by the probes listed in Table 3, and that the effectiveness of the particular probes chosen will vary, amongst other things, according to the platform used to measure transcript abundance and the hybridization conditions employed. It will therefore be appreciated that probes targeting different regions of the transcript may also be used in accordance with the present invention.

In other suitable embodiments of the invention, the target molecule for the biomarker may be a protein, and the binding partner is selected from the group consisting of antibodies, antibody fragments and aptamers.

Polynucleotides encoding any of the specific binding partners of target molecules for biomarkers of the invention recited above may be isolated and/or purified nucleic acid molecules and may be RNA or DNA molecules.

Throughout, the term “polynucleotide” as used herein refers to a deoxyribonucleotide or ribonucleotide polymer in single- or double-stranded form, or sense or anti-sense, and encompasses analogues of naturally occurring nucleotides that hybridize to nucleic acids in a manner similar to naturally occurring nucleotides. Such polynucleotides may be derived from Homo sapiens, or may be synthetic or may be derived from any other organism.

Commonly, polypeptide sequences and polynucleotides used as binding partners in the present invention may be isolated or purified. By “purified” it is meant that they are substantially free from other cellular components or material, or culture medium. “Isolated” means that they may also be free of naturally occurring sequences which flank the native sequence, for example in the case of nucleic acid molecule, isolated may mean that it is free of 5′ and 3′ regulatory sequences.

In a preferred embodiment the nucleic acid is mRNA or cDNA. There are numerous suitable techniques known in the art for the quantitative measurement of RNA transcript levels in a given biological sample. These techniques include but are not limited to; “Northern” RNA blotting, Real Time Polymerase Chain Reaction (RTPCR), Quantitative Polymerase Chain Reaction (qPCR), digital PCR (dPCR), multiplex PCR, Reverse Transcription Quantitative Polymerase Chain Reaction (RT-qPCR), branched DNA signal amplification or by high-throughput analysis such as hybridization microarray, Next Generation Sequencing (NGS) or by direct mRNA quantification, for example by “Nanopore” sequencing. Alternatively, “tag based” technologies may be used, which include but are not limited to Serial Analysis of Gene Expression (SAGE). Suitable techniques also include nCounter™ systems of NanoString Technologies™, zip coding, and targeted hybridization and sequencing. Commonly, the levels of biomarker mRNA transcript in a given biological sample may be determined by hybridization to specific complementary nucleotide probes on a hybridization microarray or “chip”, by Bead Array Microarray technology or by RNA-Seq where sequence data is matched to a reference genome or reference sequences.

In a preferred embodiment, where the nucleic acid is RNA, the present invention provides methods wherein the levels of biomarker transcript(s) will be determined by PCR. Preferably mRNA and ncRNA transcript abundance will be determined by qPCR, dPCR or multiplex PCR. More preferably, transcript abundance will be determined by multiplex-PCR. Nucleotide primer sequences may be designed to any sequence region of the biomarker transcripts (accession numbers listed in Table 3) or a variant thereof. The person skilled in the art will appreciate that equally effective primers can be designed to different regions of the transcript or cDNA of biomarkers listed in Table 3, and that the effectiveness of the particular primers chosen will vary, amongst other things, according to the platform used to measure transcript abundance, the biological sample and the hybridization conditions employed. It will therefore be appreciated that primers targeting different regions of the transcript may also be used in accordance with the present invention. However, the person skilled in the art will recognise that in designing appropriate primer sequences to detect biomarker expression, it is required that the primer sequences be capable of binding selectively and specifically to the cDNA sequences of biomarkers corresponding to the nucleotide accession numbers listed in Table 3 or fragments or variants thereof.

Many different techniques known in the art are suitable for detecting binding of the target molecule sequence and for high-throughput screening and analysis of protein interactions. According to the present invention, appropriate techniques include (either independently or in combination), but are not limited to; co-immunoprecipitation, bimolecular fluorescence complementation (BiFC), dual expression recombinase based (DERB) single vector system, affinity electrophoresis, pull-down assays, label transfer, yeast two-hybrid screens, phage display, in vivo crosslinking, tandem affinity purification (TAP), ChIP assays, chemical crosslinking followed by high mass MALDI mass spectrometry, strep-protein interaction experiment (SPINE), quantitative immunoprecipitation combined with knock-down (QUICK), proximity ligation assay (PLA), bio-layer interferometry, dual polarisation interferometry (DPI), static light scattering (SLS), dynamic light scattering (DLS), surface plasmon resonance (SPR), fluorescence correlation spectroscopy, fluorescence resonance energy transfer (FRET), isothermal titration calorimetry (ITC), microscale thermophoresis (MST), chromatin immunoprecipitation assay, electrophoretic mobility shift assay, pull-down assay, microplate capture and detection assay, reporter assay, RNase protection assay, FISH/ISH co-localization, microarrays, microsphere arrays or silicon nanowire (SiNW)-based detection. Where biomarker protein levels are to be quantified, preferably the interactions between the binding partner and biomarker protein will be analysed using antibodies with a fluorescent reporter attached.

In certain embodiments of the invention, the expression level of a particular biomarker may be detected by direct assessment of binding of the target molecule to its binding partner. Suitable examples of such methods in accordance with this embodiment of the invention may utilise techniques such as electro-impedance spectroscopy (EIS) to directly assess binding of binding partners (e.g. antibodies) to target molecules (e.g. biomarker proteins).

In certain embodiments of the present invention the binding partner may be an antibody, or antibody fragment, and the detection of the target molecules utilises an immunological method. In certain embodiments of the methods or devices, the immunological method may be an enzyme-linked immunosorbent assay (ELISA) or utilise a lateral flow device.

A method of the invention may further comprise quantification of the amount of the target molecules indicative of expression of the biomarkers that is present in the patient sample. Suitable methods of the invention, in which the amount of the target molecule present has been quantified, and the volume of the patient sample is known, may further comprise determination of the concentration of the target molecules present in the patient sample which may be used as the basis of a qualitative assessment of the patient's condition, which may, in turn, be used to suggest a suitable course of treatment for the patient.

### Reporter Moieties

In preferred embodiments of the present invention the expression levels of the protein in a biological sample may be determined. In some instances, it may be possible to directly determine expression, e.g. as with GFP or by enzymatic action of the protein of interest (POI) to generate a detectable optical signal. However, in some instances it may be chosen to determine physical expression, e.g. by antibody probing, and optionally rely on separate test to verify that physical expression is accompanied by the required function.

In preferred embodiments of the invention, the expression levels of a particular biomarker will be detectable in a biological sample by a high-throughput screening method, for example, relying on detection of an optical signal, for instance using reporter moieties. For this purpose, it may be necessary for the specific binding partner to incorporate a tag, or be labelled with a removable tag, which permits detection of expression, or alternatively for a second binding partner to be used which includes a tag and is specific for the first binding partner (where the first binding partner is specific for the target molecule). Such a tag may be, for example, a fluorescence reporter molecule translationally-fused to the protein of interest (POI), e.g. Green Fluorescent Protein (GFP), Yellow Fluorescent Protein (YFP), Red Fluorescent Protein (RFP), Cyan Fluorescent Protein (CFP) or mCherry. Such a tag may provide a suitable marker for visualisation of biomarker expression since its expression can be simply and directly assayed by fluorescence measurement in vitro or on an array. Alternatively, it may be an enzyme which can be used to generate an optical signal. Tags used for detection of expression may also be antigen peptide tags or quantum dots, which may be used to tag proteins such as antibodies. Similarly, reporter moieties may be selected from the group consisting of fluorophores; chromogenic substrates; and chromogenic enzymes. Other kinds of label may be used to mark a nucleic acid binding partner including organic dye molecules, radiolabels and spin labels which may be small molecules.

Preferably, the levels of a biomarker or several biomarkers will be quantified by measuring the specific hybridization of a complementary nucleotide probe to the target molecule for the biomarker of interest under high-stringency or very high-stringency conditions.

Preferably, probe-target molecule hybridization will be detected and quantified by detection of fluorophore-, silver-, or chemiluminescence-labelled probes to determine relative abundance of biomarker nucleic acid sequences in the sample. Alternatively, levels of biomarker mRNA or ncRNA transcript abundance can be determined directly by RNA sequencing or nanopore sequencing technologies.

The methods or devices of the invention may make use of target molecules selected from the group consisting of: the biomarker protein; and nucleic acid encoding the biomarker protein. Where the target molecule is the biomarker protein, it is preferred that the binding partner is an antibody or antibody fragment and immunofluorescence is used to detect binding; methods of detecting bound antibodies and antibody fragments are well known in the art and may include the use of a tag attached to the antibodies or fragments themselves, and/or the use of additional antibodies that have such a detectable, e.g. fluorescent, tag and are specific for the primary antibody or fragment.

### Nucleotides and Hybridization Conditions

Throughout, the term “polynucleotide” as used herein refers to a deoxyribonucleotide or ribonucleotide polymer in single- or double-stranded form, or sense or anti-sense, and encompasses analogues of naturally occurring nucleotides that hybridize to nucleic acids in a manner similar to naturally occurring nucleotides.

Exemplary probe sequences are provided in Table 3, although it will be appreciated that minor variations in these sequences may work. The person skilled in the art would regard it as routine to design nucleotide probe sequences may be designed to any sequence region of the biomarker transcripts (accession numbers listed in Table 3) or a variant thereof. This is also the case with nucleotide primers used where detection of expression levels is determined by PCR-based technology. Nucleotide probe sequences, for example, may include, but are not limited to those listed in Table 3. The person skilled in the art will appreciate that equally effective (and in some cases more beneficial) probes can be designed to different regions of the transcript than those targeted by the probes listed in Table 3, and that the effectiveness of the particular probes chosen will vary, amongst other things, according to the platform used to measure transcript abundance and the hybridization conditions employed. It will therefore be appreciated that probes targeting different regions of the transcript may also be used in accordance with the present invention.

Of course the person skilled in the art will recognise that in designing appropriate probe sequences to detect biomarker expression, it is required that the probe sequences be capable of binding selectively and specifically to the transcripts or cDNA sequences of biomarkers corresponding to the nucleotide accession numbers listed in Table 3 or fragments or variants thereof. The probe sequence will therefore be hybridizable to that nucleotide sequence, preferably under stringent conditions, more preferably very high stringency conditions. The term “stringent conditions” may be understood to describe a set of conditions for hybridization and washing and a variety of stringent hybridization conditions will be familiar to the skilled reader. Hybridization of a nucleic acid molecule occurs when two complementary nucleic acid molecules undergo an amount of hydrogen bonding to each other known as Watson-Crick base pairing. The stringency of hybridization can vary according to the environmental (i.e. chemical/physical/biological) conditions surrounding the nucleic acids, temperature, the nature of the hybridization method, and the composition and length of the nucleic acid molecules used. Calculations regarding hybridization conditions required for attaining particular degrees of stringency are discussed in Sambrook et al. (2001, Molecular Cloning: A Laboratory Manual, Cold Spring Harbor Laboratory Press, Cold Spring Harbor, N.Y.); and Tijssen (1993, Laboratory Techniques in Biochemistry and Molecular Biology-Hybridization with Nucleic Acid Probes Part I, Chapter 2, Elsevier, N.Y.). The Tm is the temperature at which 50% of a given strand of a nucleic acid molecule is hybridized to its complementary strand.

In any of the references herein to hybridization conditions, the following are exemplary and not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
High Stringency (Allows Sequences that Share at Least 80% Identity to Hybridize)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
Low Stringency (Allows Sequences that Share at Least 50% Identity to Hybridize)
Hybridization: 6×SSC at RT to 55° C. for 16-20 hours
Wash at least twice: 2×-3×SSC at RT to 551C for 20-30 minutes each.

### Diagnostic Devices and Kits

The invention also provides an assay device for use in the above methods, the device comprising: a) a loading area for receipt of a biological sample; b) binding partners specific for target molecules representative of expression of at least two biomarkers selected from the group listed in Table 1; and c) detection means to detect the levels of said target molecules present in the sample.

Suitably the device comprises specific binding partners for amplifying the target molecules of the biomarkers. Suitable binding partners and associated reporter moieties for use in the devices and kits of the invention are described above. A variety of suitable PCR amplification-based technologies are well known in the art.

The binding partners are preferably nucleic acid primers adapted to bind specifically to the mRNA or cDNA transcripts of the biomarkers, or one or more labelled antibodies that binds to one of the biomarker proteins, as discussed above. Suitably, the kit may comprise a combination of nucleic acid primers and antibodies, for example nucleic acid primers may be provided in the kit for analysing the levels of some biomarkers of Table 1, whilst antibodies may be provided in the kit for analysing the levels of some other biomarkers of Table 1.

The detection means suitably comprises means to detect a signal from a reporter moiety, e.g. a reporter moiety as discussed above.

The device is adapted to detect and quantify the levels of said biomarkers present in the biological sample.

The invention provides kits for use in the above methods, the kits comprising binding partners capable of binding to target molecules representative of expression of at least two biomarkers selected from the group listed in Table 1. Preferably the kits further comprise indicators capable of indicating when said binding occurs.

Preferably the kits and devices comprise binding partners capable of binding to target molecules representative of expression of at least three biomarkers, for example at least 4, 5, 8, 10, 12, 15, 18, 20, 21, 23, 25, 28, 30, 32, 35, 36, or 37 biomarkers corresponding to genes in the list of Table 1. Preferably the kits and devices comprise binding partners capable of binding to target molecules representative of expression of at least 5 genes in the list of Table 1, at least 15, at least 21, or at least 30 genes in the list of Table 1. Further preferably the kits and devices comprise binding partners capable of binding to target molecules representative of expression of at least 5 genes in the list of Table 2, at least 10, at least 15, or at least 20 genes in the list of Table 1. The kits and devices may comprise binding partners capable of binding to target molecules representative of expression of all the biomarkers in Table 2, and optionally all the biomarkers in Table 1.

PCR applications are routine in the art and the skilled person will be able to select appropriate polymerases, buffers, reporter moieties and reaction conditions.

The binding partners are preferably nucleic acid primers adapted to bind specifically to the mRNA, ncRNA, or cDNA transcripts of biomarkers, as discussed above. The nucleic acid primers may be provided in a lyophilized or reconstituted form, or may be provided as a set of nucleotide sequences. In one embodiment, the primers are provided in a microplate format, where each primer set occupies a well (or multiple wells, as in the case of replicates) in the microplate. The microplate may further comprise primers sufficient for the detection of one or more housekeeping genes as a positive control. The kit may further comprise reagents and instructions sufficient for the amplification of expression products from the biomarkers.

As well as the binding partners for the target molecules of the biomarkers, the devices and kits may further comprise binding partners capable of binding to target molecules representative of expression of additional genes. For example, such genes may be “housekeeping genes”, which can act as a positive control and/or to normalize expression across samples, and/or such genes may give an indication of the concentration of the monocyte population within the biological sample.

Preferably said devices and kits provide binding partners capable of binding to target molecules representative of expression of less than 100, 75, 50, 40, or 30 genes, including the biomarkers and any housekeeping or other control genes.

The kit can optionally comprise instructions for carrying out the analysis required for the methods of the invention.

### BRIEF DESCRIPTION OF THE FIGURES

The invention will now be described in detail with reference to a specific embodiment and with reference to the accompanying figures, in which:

FIG. 1. Sorting strategy for tissue macrophages and TAMs and analysis of CD163 expression.

A) Gating strategy for tissue macrophages and TAMs; macrophages were defined as CD45+CD3/56/19-CD11b+CD14+CD163+.

B) (Left) Representative histogram of macrophage CD163 expression; black histogram=CD163 staining, grey histogram=Fluorescence minus one (FMO) control; (Right) Representative histogram of macrophage CD163 expression in Br-MR (n=5) compared to Br-TAM (n=5). Data are expressed as Geometric Mean (Mean±SEM).

FIG. 2. TAMs from breast and endometrial cancers exhibit cancer-specific transcriptional profiles.

A) PCA plot of N=13,668 genes expressed in breast tissue resident macrophages (Br-RM, triangles, N=4) and breast cancer TAMs (Br-TAM, circles, N=4). B) Hierarchical clustering of all DEGs between Br-RM and Br-TAM. Expression values are Z score-transformed. Samples were clustered using complete linkage and Euclidean distance. C) Gene ontology analysis of DEGs between Br-TAM and Br-RM (five GO groups at bottom of chart=down-regulated genes, six GO groups at top of chart=upregulated genes). D) Bar plot of selected DEGs in Br-TAM (FDR<=0.05). E) PCA plot of N=13,739 genes expressed in endometrial tissue resident macrophages (En-RM, N=5) from healthy individuals and endometrial cancer TAMs (En-TAM, N=9). F) Hierarchical clustering of all DEGs between En-RM and En-TAM. Expression values are Z score-transformed. Samples were clustered using complete linkage and Euclidean distance. G) Gene ontology analysis of DEGs between En-TAM and En-RM (nine GO groups at bottom of chart=down-regulated genes, three GO groups at top of chart=upregulated genes). H) Bar plot of selected DEGs in En-TAM (FDR<=0.05).

FIG. 3. Comparison of TAMs and resident macrophages.

A) (Left) PCA plot of N=14,229 expressed genes in Br-TAM (n=4) and En-TAM (n=9). (Right) Hierarchical clustering of all DEGs between Br-TAM and En-TAM. Expression values are Z score-transformed. Samples were clustered using complete linkage and Euclidean distance.

B) (Left) PCA plot of N=13,907 expressed genes in Br-RM (n=4) and En-RM (n=5). (Right) Hierarchical clustering of all DEGs between Br-RM against En-RM. Expression values are Z score-transformed. Samples were clustered using complete linkage and Euclidean distance.

C) Enrichment analysis of M1-like (left) and M2-like (right) macrophage signature (10) in Br-TAM. Black bars represent the position of M1-like or M2-like genes in the ranked list of Br-TAM expressed genes together with the running enrichment score.

D) Enrichment analysis of M1-like (left) and M2-like (right) macrophage signature in En-TAM. Black bars represent the position of M1-like or M2-like genes in the ranked list of Br-TAM expressed genes together with the running enrichment score.

FIG. 4. TAM metagene signature analysis on breast cancer patients.

(A and B) Box plot showing TAM signature score stratified by the CSF1 signature (A) and across breast cancer subtypes in Cohort 3 (n=47) (B). (C) TAM signature score across PAM50 molecular subtypes in the METABRIC cohort (n=1350). (D and E) Disease-specific survival of the METABRIC cohort according to the TAM signature expression (D) and the MAC signature expression (E). Boxplots depict the first and third quartiles, with the median shown as a solid line inside the box and whiskers extending to 1.5 interquartile range from first and third quartiles. (A-C) one-way ANOVA with Tukey's post-hoc multiple comparisons test (***p<0.0001). (D) p value is based on Wald test.

### EXPERIMENTAL RESULTS

### 1. Materials and Method

### Patient and Control Samples

All study protocols were approved by the IRB of the Albert Einstein Medical College (Bronx, N.Y., USA), by The University of Edinburgh (Edinburgh, UK) and Duke University (Durham, N.C.) ethics committees as appropriate. Informed consent was obtained from all human subjects included in this study.

Cohort 1: Breast cancer tissue (0.1-1 grams) and endometrial cancer tissue (0.1-1 grams) was obtained from Montefiore Medical Center, NY, USA. Normal breast tissue from mammoplasty reduction surgeries (25-50 grams) was obtained from the Human Tissue Procurement Facility (HTPF), Ohio State University, USA; normal/benign endometrial tissue (1-2 grams) was obtained after surgery for conditions unrelated to cancer from Montefiore Medical Center, NY, USA.

Cohort 2: Cancer tissue (0.1-1 grams) was obtained from breast cancer patients from NHS, Edinburgh, Scotland, UK. Normal/benign breast tissue (0.5-1 grams) from patients with benign conditions was obtained from NHS, Edinburgh, Scotland, UK.

Cohort 3: Breast cancer tissue was obtained by Duke University, Durham N.C., USA. Pathologically the breast cancer patients consisted of invasive breast cancers with either node⁻ or node⁺ disease. Patients had biopsy-confirmed invasive tumors of at least 1.5 cm at diagnosis. Tumor samples were shipped on ice to Oregon Health & Science University Hospital (OHSU) for immune and genomic assays.

The exclusion criteria for all cancer patients at baseline included systemic metastatic disease, any inflammatory disorder, and active infection or immunocompromised status not related to cancer. All the patients recruited were chemotherapy and radiotherapy naive before collection.

### Clinical Details of Cohort 1 and 2

| | | Cohort 1 | | Cohort 2 |
|---|---|---|---|---|
| Variables | Cohort1 | CTR | Cohort 2 | CTR |
| No of patients | 60 | 46 | 65 | 52 |
| Age, Years, median | 65 | 48 | 60 | 45 |
| (range) | (42-81) | (38-62) | (23-86) | (27-72) |
| Gender | | | | |
| Male | 0/60, | 0/46, | 0/65, | 0/52 |
| Female | 60/60 | 46/46 | 65/65 | 52/52 |
| Type of cancer | | | | |
| Breast | 40/60, | n/a | 65/65, | n/a |
| Endometrium | 20/60 | n/a | 0/65 | n/a |
| Breast Cancer Grade | 1 (16/40) | n/a | 1(18/65) | n/a |
| (invasive) | | | | |
| Breast Cancer Grade | 2 (11/40) | | 2 (25/65) | |
| (invasive) | | | | |
| Breast Cancer Grade | 3(13/40) | n/a | 3 (22/65) | n/a |
| (invasive) | | | | |
| Breast Cancer ER⁺ | 32/40 | n/a | 65/65 | n/a |
| Breast Cancer PR⁺ | 28/40 | n/a | 7/65 | n/a |
| Breast Cancer HerER2+ | 8/40 | n/a | 1/65 | n/a |
| Breast Cancer | 8/40 | n/a | 0/65 | n/a |
| ER⁻/PR⁻/HerER2− | | | | |
| Endometrial Cancer Grade | 1 (4/20) | n/a | n/a | n/a |
| (invasive) | | | | |
| Endometrial Cancer Grade | 2(5/20) | n/a | 0/65 | n/a |
| (invasive) | | | | |
| Endometrial Cancer Grade | 3(11/20) | n/a | n/a | n/a |
| (invasive) | | | | |
| Endometrial Cancer Type 1 | 10/20 | n/a | n/a | n/a |
| Endometrial Cancer Type 2 | 10/20 | n/a | n/a | n/a |

### Clinical Details of Cohort 3

| | | Tumor | | | Node | | | | |
|---|---|---|---|---|---|---|---|---|---|
| | | Size | | | Status | | ER | PR | Her2 |
| Age | Race | (cm) | Grade | Histology | (pos/total) | Node | Status | Status | Status |
| 60 | W | 2.4 | 1 | Lobular | 0/3 | − | 8 | 7 | 1 + |
| | | | | | | | (100) | (90) | (20) |
| 70 | W | 3.5 | 2 | Lobular | 13/15 | + | 8 | 6-7 | 2 + |
| | | | | | | | (100) | (66) | |
| 71 | W | 2.1 | 3 | Ductal | 0/2 | − | 0 | 0 | 2 + |
| 42 | AA | 15.9 | 3 | Ductal | 8/21 | + | 7-8 | 6 | 1 + |
| | | | | | | | (95) | (66) | (80) |
| 55 | AA | 3.7 | 2 | Ductal | 1/12 | + | 0 | 0 | 1 + |
| | | | | | | | | | (10) |
| 82 | Other | 3 | 2 | Ductal | 5/15 | + | 8 | 3-4 | 2 + |
| | | | | | | | (100) | (1) | (70) |
| 49 | W | 1.9 | 2 | Lobular | 0/6 | − | 8 | 8 | 1 + |
| | | | | | | | (100) | (100) | (10) |
| 43 | W | 1.5 | 2 | Ductal | 0/4 | − | 6 | 3 | 1 + |
| 68 | W | 4.2 | 3 | Metaplastic | 0/6 | − | 0 | 0 | 0 |
| 50 | W | 3.7 | 2 | Ductal | 4/27 | + | 0 | 0 | 0 |
| 34 | W | 2.5 | 2 | Solid | 0/1 | − | 7-8 | 0 | 2 + |
| | | | | | | | (90) | | (30) |
| 42 | AA | 3.5 | 2 | Ductal | 14/16 | + | 0 | 0 | 2 + |
| | | | | | | | | | (20) |
| 46 | W | 1.5 | 3 | Ductal | 0/5 | − | 7-8 | 8 | 3 + |
| | | | | | | | (89-90) | (100) | (100) |
| 76 | | 5.1 | 2 | Ductal | 0/3 | − | 0 | 0 | 2 + |
| 74 | | 0.8 | 2 | Ductal | 1/15 | + | 1 + (1) | 0 | 2 + |
| 53 | W | 3 | 3 | Ductal | 4/29 | + | 0 | 0 | 3 + |
| | | | | | | | | | (100) |
| 73 | | 4.2 | 2 | Ductal | 4/14 | + | 3 + | 2-3 + | 3 + |
| | | | | | | | (100) | (50) | |
| 66 | | 9.6 | 3 | Ductal | 4/4 | + | 0 | 0 | 0 |
| 56 | | 2.6 | 3 | Ductal | 0/7 | − | 3 + | 3 + | 0 |
| | | | | | | | (>95) | (>90) | |
| 49 | | 4.3 | 3 | Ductal | 0/3 | − | 3 + | 0 | 2 + |
| | | | | | | | (90) | | |
| 36 | | 1.9 | 1 | Ductal | 4/32 | + | 3 + | 3 + | 0 |
| | | | | | | | (100) | (100) | |
| 61 | | 4.8 | 3 | Ductal | 1/17 | + | 3 + | 3 + | 2 + |
| | | | | | | | (99) | (60) | |
| 78 | | 2.2 | 3 | Ductal | 0/1 | − | 3 + | 1-3 + | 3 + |
| | | | | | | | (30) | (40) | |
| 54 | | 7 | 2 | Lobular | 5/24 | + | 3 + | 3 + | 0 |
| | | | | | | | (30) | (30) | |
| 78 | | 2.2 | 3 | Ductal | 0/0 | NP | 3 + | 0 | 2 + |
| | | | | | | | (100) | | |
| 65 | | 9 | 2 | Ductal | 8/36 | + | 1-3 + | 1-3 + | − |
| | | | | | | | (90) | (60) | |
| 65 | | 1.1 | 2 | Lobular | 0/1 | − | 3 + | 1-3 + | − |
| | | | | | | | (100) | (60) | |
| 59 | | 7 | 2 | Lobular | 4/17 | + | 3 + | 1 + | 0 |
| | | | | | | | (100) | (10) | |
| 24 | | 2.8 | 3 | Ductal | 0/17 | − | 0 | 0 | 2 + |
| 39 | | 9.9 | 2 | Ductal | 05/13 | + | 3 + | 0 | 2 + |
| | | | | | | | (>95) | | |
| 38 | | 9.2 | 2 | Ductal | 01/15 | + | 0 | 0 | 3 + |
| 51 | | 4.2 | 3 | Ductal | 0/5 | − | 2 + | 0 | 2 + |
| | | | | | | | (20) | | |
| 71 | | 3 | 1 | Lobular | 0/3 | − | 3 + | 0 | 0 |
| | | | | | | | (>95) | | |
| 47 | | 2.1 | 1 | Ductal | 0/2 | − | 3 + | 3 + | 0 |
| | | | | | | | (90) | (70) | |
| 47 | | 5.3 | 1 | Ductal | 4/12 | + | 3 + | 3 + | 0 |
| | | | | | | | (95) | (50) | |
| 60 | | 3.7 | 2 | Ductal | 2/26 | + | 3 + | 1 | 2 + |
| | | | | | | | (>90) | (10) | |
| 33 | W | 10 | 3 | DCIS | 0/5 | − | 3 | 3 | − |
| | | | | (Comedo) | | | (10) | (<1) | |
| 70 | W | 2.2 | 2 | Ductal | 4/20 | + | 1 | 2 | 0 |
| | | | | | | | (95) | (99) | |
| 70 | W | 4.6 | 2 | Lobular | 5/16 | + | 8 | 8 | 0 |
| | | | | | | | (100) | (100) | |

| | | |
|---|---|---|
| 47 | W | Prophylactic Mastectomy |
| 37 | | Prophylactic Mastectomy |
| 41 | | Prophylactic Mastectomy |

### Isolation of Human Tissue Macrophages.

Cancer tissue and normal endometrial tissue were washed with Phosphate Buffer Saline (PBS) in a petri dish and tissue was chopped into small fragments with a razorblade on ice. The sample was transferred to a 15-50 ml tube according to size and Liberase enzymes TL (14 U/mL) and DL (28 U/mL) (Roche) and DNAse (15 mg/mL) (Roche) were added in serum-free PBS. Tissue was digested at 37° C. on a rotating wheel for 1-18 hr depending on tissue weight; at the end of digestion the cell suspension was filtered using a 100 μm cell strainer and PBS 1% w/v Bovine Serum Albumin (BSA, Sigma-Aldrich) was added in order to interrupt the digestion process. Cells were centrifuged at 400 RCF for 5 min at 4° C. in a swinging bucket rotor. The pellet was re-suspended in PBS, 1% w/v BSA and cells counted and stained for FACS sorting or analysis. Macrophages were sorted using the antibodies CD45 AlexaFluor-700, CD3 PE-Cy5, CD56 PE-Cy5, CD19 PE-Cy5, CD14 FITC, CD11b PE-Cy7, CD163 APC (11).

### Flow Cytometry Sorting and Analysis

Blocking of Fc receptors was performed by incubating samples with 10% v/v human serum (Sigma Aldrich) for 1 hr on ice. For cytofluorimetric analysis 5×10⁵ cells were stained in a final volume of 100 μL using the following antibodies at 1:100 dilutions: CD45 PE-Texas Red, CD3-, CD56-, CD19-BV711, CD11b BV605, CD14 BV510, CD16 EF450, CX3CR1 FITC, HLA-DR BV650, CCR2 PE-Cy7 (Biolegend). For macrophage sorting cells were stained and antibody concentration was scaled up based on cell number; cells were stained with the following antibodies at 1:100 dilutions: CD45-AlexaFluor 700, CD3-, CD56-, CD19-PE-Cy5, CD14 FITC, CD11b PE-Cy7, CD16 PE-Texas Red, CD163 APC (Biolegend). Cancer cell lines were stained for the 5 CCL8 receptors with the following antibodies: CCR1 PE, CCR2 PE-Cy7, CCR3 FITC, CCR5 PE, CCR8 PE (Biolegend). Cells were incubated in the dark for 1 hr on ice; after washing with PBS 1% w/v BSA (analysis) or PBS 0.1% w/v BSA (sorting) cells were filtered and re-suspended in the appropriate buffer before analysis or sorting. Cytofluorimetric analysis was performed using a 6-laser Fortessa flow cytometer (BD); FACS sorting was performed using FACS Ariall and FACS Fusion sorters (BD). Cell sorting was performed at 40C in 1.5 ml RNAse and DNAse free tubes (Simport, Canada) pre-filled with 750 μl of PBS 0.1% w/v BSA; at the end of each isolation a sorting purity check was performed. A minimum of 5,000 events in the monocyte/macrophage gate was acquired for cytofluorimetric analysis. Results were analyzed with Flowjo (Treestar) or DIVA software (BD)

### RNA Sequencing and Bioinformatic Analysis

Immediately after sorting all the samples were centrifuged at 450 RCF for 10 min at 4 C. The cell pellet was resuspended in 350 uL of RLT lysis buffer and RNA extracted with RNAeasy Microkit (Qiagen) according to manufacturer's instructions. RNA quantity was determined by QUBIT (Invitrogen); total RNA integrity was assessed by Agilent Bioanalyzer and the RNA Integrity Number (RIN) was calculated; samples that had a RIN>7 were selected for RNA amplification and sequencing. RNA was amplified with Ovation RNAseq Amplification kit v2 (Nugen) according to manufacturer's instructions; amplified RNA was sent to Albert Einstein Genomic Facility (https://www.einstein.yu.edu/departments/genetics/resources/genomics-core.aspx) or BGI (Philadelphia; http://en.genomics.cn/navigation/show_navigation?nid=271) where library preparation, fragmentation and paired-end multiplex sequencing were performed (HIseq 2000 and 2005, Illumina). All samples were processed and randomly assigned to lanes without knowledge of clinical identity to avoid bias and batch effects.

### Sequencing Alignment and Quantification

FastQ files of 2×100 bp paired-end reads were quality checked using FastQC. Samples were filtered for low quality reads (Phred score>=20) and adapters were removed when necessary using Cutadapt. Quality controlled reads were then aligned to the human reference genome (GRCh37/hg19) using STAR aligner (version 2.3). Quantification of genes was performed using the count function of HTseq. Reads were counted at the gene level and the unstranded option was used (-s no).

### Statistical Analysis for Differentially Expressed Genes

All statistical calculations were performed in R programming language (version 3.2.3). For macrophage samples, genes with count per million (CPM) reads>1 in at least N samples (N number of the fewest replicates of a condition) was retained. Gene expression levels were normalized using the Trimmed Mean of M-values (TMM) method using the calcNormFactorso function and log₂ transformed using the cpmo function from the EdgeR package in R. Differential expression analysis was performed with sample quality weights using the package limma-voom package in R. Differential expression analysis was performed using the limma package. Significantly differentially expressed genes (DEGs) were selected with controlled False Positive Rate (B&H method) at 5% (FDR<=0.05). Up-regulated genes were selected at a minimum log 2 fold change of 1.5 and down-regulated genes at a minimum log 2 fold change of −1.5. PCA plots were drawn using the TMM/log₂ transformed (macrophages) values on expressed genes. Heatmaps were drawn on the normalized expression matrix using the pheatmap package in R. Euclidean distance and complete linkage were used for hierarchical clustering. Venn diagrams were constructed based on the overlapping differentially expressed transcripts (FDR<=0.05, Log₂FC more or less than 1.5/−1.5).

### Enrichment and Pathway Analysis

Gene set enrichment analysis was performed using the gseao function from phenoTest package in R. The function is used to compute the enrichment scores and simulated enrichment scores for each variable and signature. For our analysis, the logscale variable was set to false, as the log₂ transformed expression values were fed into the function and 20,000 simulations were used (B=20,000). The Database for Annotation, Visualization and Integrated Discovery (DAVID) functional annotation tool was used for gene ontology and pathway (KEGG and Reactome) analysis on the list of differentially expressed genes (FDR<=0.05, Log₂FC more or less than 1.5/−1.5). Important GO terms and pathways were selected based on an FDR<=0.05.

### Publicly Available Datasets

The following publicly available datasets were used in this study:

Breast cancer:
a) Karnoub et al. (GSE8977) (12): total of 22 samples coming from breast ductal carcinoma-in-situ (DCIS) patients (n=15) and invasive ductal (IDC) breast cancer patients (n=7) were downloaded from GEO. Samples were processed and normalized using the robust Multi-Array average expression measure (RMA) from the affy package in R. Probes representing the same gene were averaged to a single value.
b) Finak et al. (GSE9014) (13): total of 59 samples coming from breast cancer stroma patients (n=53) and healthy controls (n=6) including updated clinical information were downloaded from GEO. Technical replicates were averaged to a single array using the averarrayso function from limma package in R. Data were then quantile normalized using the normalizeQuantileso function. Samples were annotated and probes representing the same gene were averaged to a single value.
c) METABRIC cohort (14): Microarray gene expression data and associated clinical information (n=1980) (Log₂ transformed intensity values) were downloaded from the cBioPortal for cancer genomics database (http://www.cbioportal.org/) under the study name Breast cancer. Gene expression values were quantile normalized and samples with gene expression and corresponding clinical information were selected resulting in n=1353 patients. Data were filtered for missing values and samples with molecular subtype NC were removed. The filtering resulted in n=1350 patients that were used for further analysis. For survival analysis, events were censored based on disease-related deaths (Died of disease=1; Living or Died of other causes=0).
d) Cancer cell Encyclopedia (CCLE) data: Gene expression RPKM normalized reads from breast cancer cell lines (n=57) were downloaded from the CCLE website (https://portals.broadinstitute.org/ccle).

### TAM Signature

As a starting point for the selection of the immune signature we used the upregulated genes in Br-TAM compared to Br-RM (n=553, Log₂FC>3 and FDR<0.05). This gene list of highly differentially expressed genes was filtered using the compendium of immune genes that includes 17 immune cell-specific gene sets as initially assembled by Bindea et al. and Charoentong et al. (15,16) and most recently validated by Tamborero et al. (17). After filtering, n=528 TAM related genes were selected. In order to identify the most relevant genes we used the METABRIC cohort and correlation analysis. Genes were considered coexpressed when having an absolute Pearson correlation of R>=0.5 (findCorrelationo function from the caret package in R (18). This threshold was selected in order to satisfy two main aims: a) genes with relatively high correlation would not be considered a chance event; b) selection of a relatively small number of genes in order to be suitable for gene set enrichment and survival analysis. Additionally, genes were selected based on their positive Pearson correlation (R>0.5, p<=0.05) with known TAM marker CD163, resulting in n=37 genes. Finally, we downloaded breast cancer cell line data from the CCLE database (n=57 breast cancer cell lines) in order to filter out genes expressed by tumor cells. Genes with median expression of Log₂RPKM>6 were considered expressed in tumor cells (TAM: median=0.031). This resulted in a set of 37 genes expressed by TAMs and not tumor cells or other immune-specific signatures. The TAM and macrophage signature scores were calculated as the median of expression of the TAM or macrophage signature genes using the median centered normalized values.

### Survival Analysis

For the TAM signature and SIGLEC1/CCL8 signature the summed normalized gene expression values were dichotomized based on the optimal cutoff calculated by iteratively calculating every possible expression cutoff (n−1) and selecting the value with the lowest p value (19). For the METABRIC cohort, disease-specific survival (DSS) was used as an endpoint. For the breast cancer stroma dataset, recurrence-free survival (RFS) was used as an endpoint and censored at date of last follow-up. Survival curves were estimated using the Kaplan Meier method (survival and survminer R packages). For SIGLEC1 and CCL8 single gene survival analysis, clinical risk factors such as ER status (+/−), PR status (+/−), Her2 status (+/−), histological grade (I, II or Ill), age (greater or less 55) and tumor size (greater or less than 50 mm) were used in the univariate and multivariate models. Candidate prognostic factors for RFS and DSS with a p value (Wald test) lower than 0.05 in univariate analysis were used in the multivariate analysis. Multivariate analysis was performed by fitting a Cox proportional hazard regression model. The Cox regression model was used to calculate the Hazard ratio (HR) and 95% confidence internal (CI). A p value less than 0.05 based on a Wald test was considered significant.

### RNA-Seq of Total Tissue Breast Cancer

RNA isolated from 47 breast cancer tumors (cohort 3) was utilized for RNA-seq. These RNA samples were converted into a library of cDNA fragments. Illumina sequencing adapters were added and 50 bp single end read sequence was obtained using Illumina HiSeq. Quality check was performed on these sequence reads using FastQC. PCR primers and adapters were filtered out of the sequence reads using Trimmomatic (20). Filtered reads were aligned to reference genome build hg19 using TopHat 2.0.12, a splice junction aligner (21). Aligned sequences were assembled into transcripts. Transcript abundance was estimated as Fragments Per Kilobase of exon per Million fragments mapped (FPKM), using Cufflinks 2.2.1 (22). FPKM estimates were normalized using Cuffnorm. Further data was quartile normalized and batch effects were removed using ComBat. Samples were classified into CSF1 High, Mid and Low expression groups using K-means clustering on the expression of CSF1 signature genes (23). TAM signature score was estimated from the median of expression of TAM signature genes. Samples were assigned to breast cancer subtypes based on hierarchical clustering of PAM50 genes (24). Clustering was performed using R package pheatmap_1.0.8. Correlation was used as a distance measure and average was used as clustering method.

### Statistical Analysis

Statistical significance was calculated by Student's t-test when comparing two groups or by one-way or two-way ANOVA when comparing three or more groups. A p value<0.05 was considered as statistically significant.

### Data and Software Availability

The RNA-seq data have been deposited in the GEO database under accession numbers GSE100925 and GSE117970.

### 2. Results

### Gene Expression Profiles of Tams in Human Breast and Endometrial Cancers

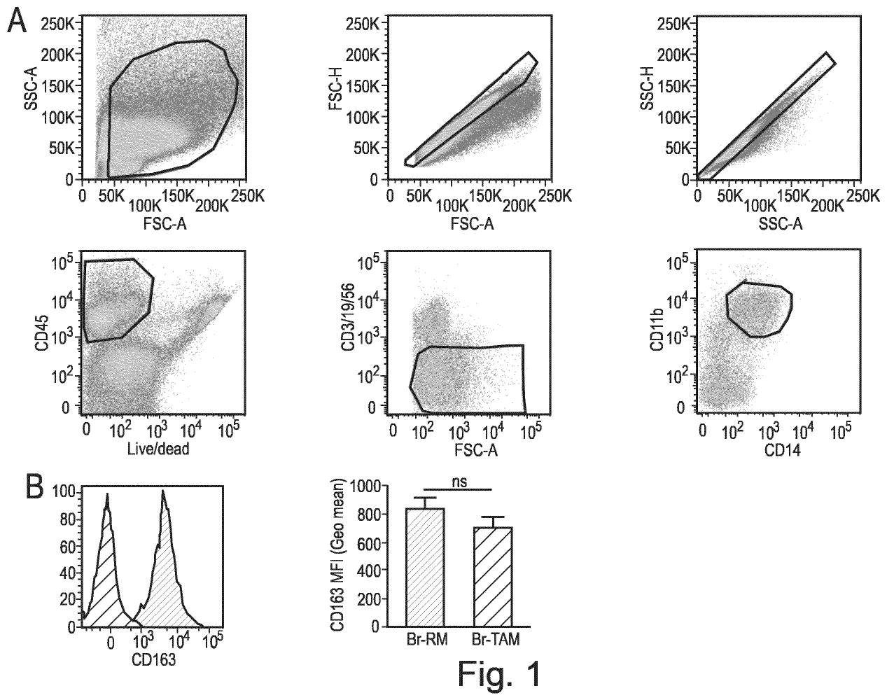

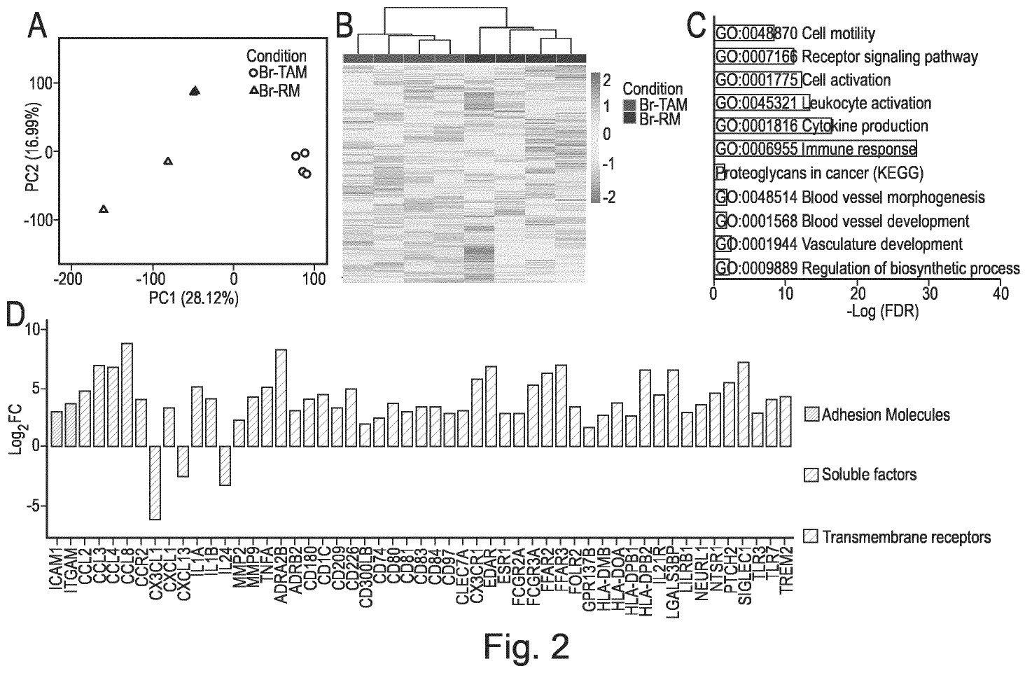

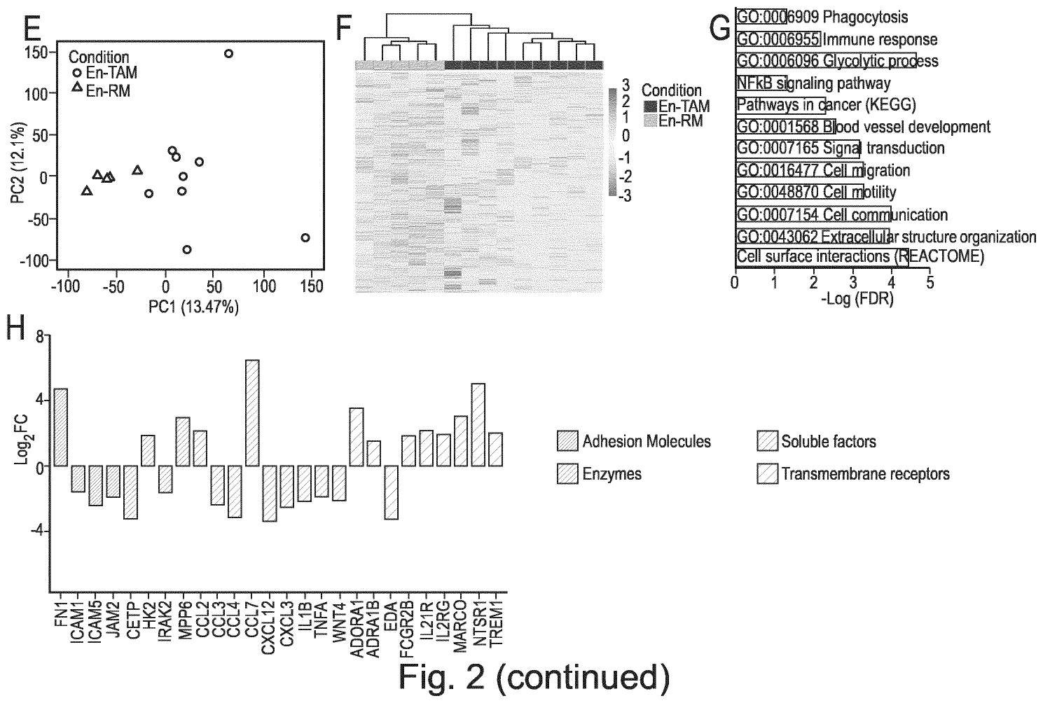

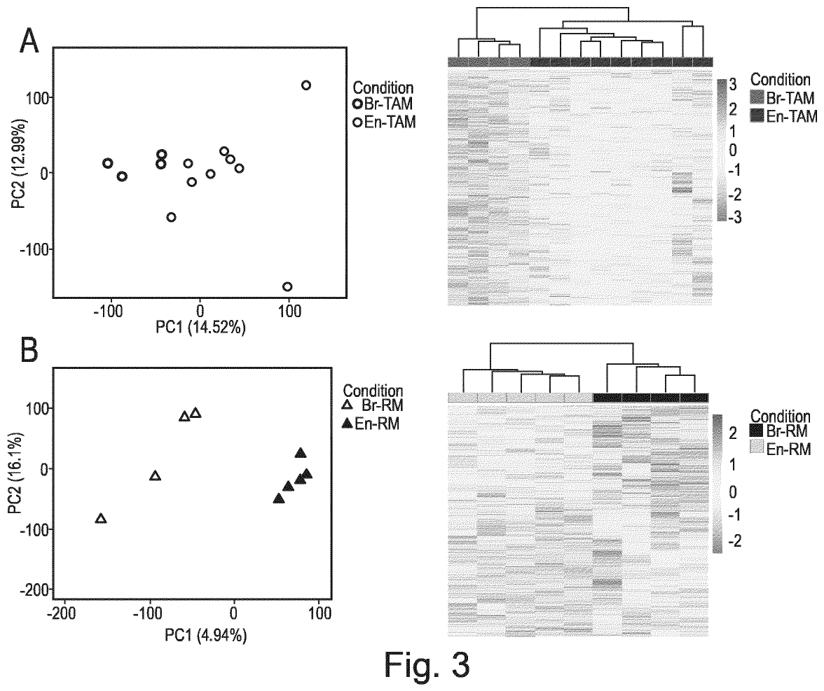

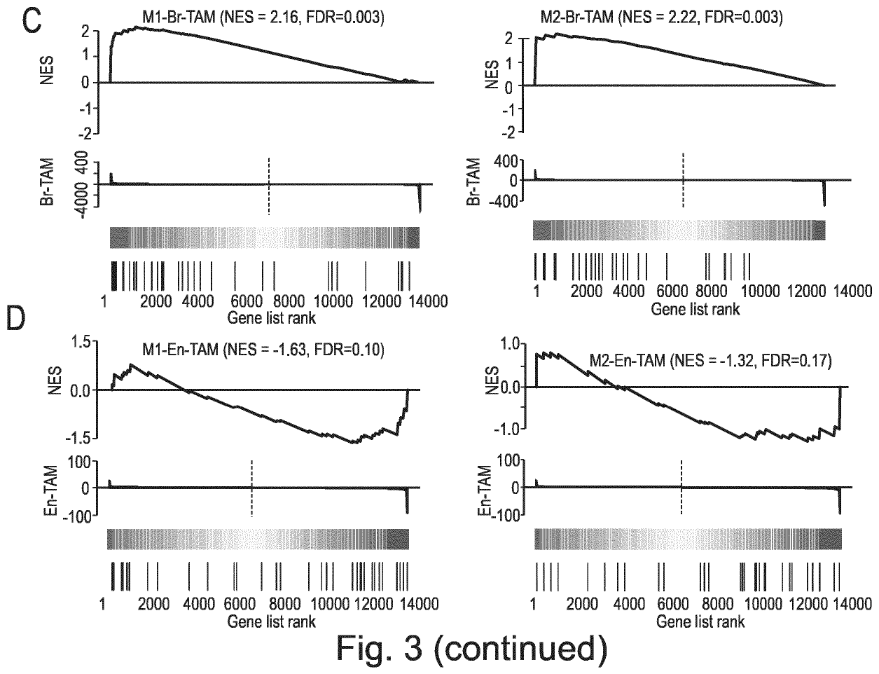

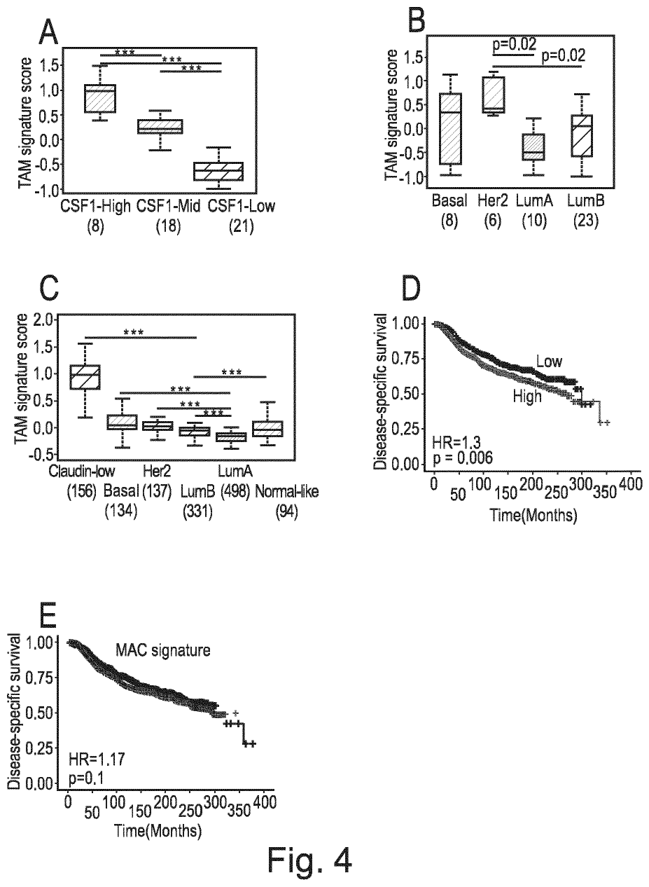

There is significant evidence showing pro-tumoral profiles of TAMs in mouse models of cancer; however, a detailed characterization of their transcriptomes and phenotypes in human cancers is still lacking. Thus, we analysed TAM transcriptomes by RNA-seq from breast and endometrial cancer in comparison to resident macrophages from homeostatic tissue after FACS sorting (FIG. 1A). PCA and hierarchical clustering revealed distinct clusters of breast tissue resident macrophages (Br-RM) and breast cancer TAMs (Br-TAM) (FIG. 2A, B). Limma DEA revealed 1873 DEGs in Br-TAM compared with Br-RM (1301 up and 572 down; FDR<=0.05). Gene ontology analysis reported several enriched GO terms such as cell motility and activation, vasculature development and immune response (FIG. 2C). Br-TAM showed increased transcript abundance of genes encoding transmembrane receptors associated with immune cell activation and antigen presentation such as MHC class II molecules, Fc receptors, T cell co-stimulatory molecules (CD80 and CD83), TLRs and Ig receptor superfamilies, and TREMs (FIG. 2D). Although in mice CD163 is often referred to as a TAM marker, we did not observe a significant difference in CD163 expression between Br-RM and Br-TAM (FIG. 1B).

PCA and hierarchical clustering revealed distinct clusters of endometrial tissue resident macrophages (En-RM) and endometrial cancer TAMs (En-TAM) (FIG. 2E, F). Limma DEA between En-RM and En-TAM identified 831 DEGs (115 up and 716 down; FDR<=0.05). Gene ontology analysis reported several enriched GO terms such as phagocytosis, immune response, cell communication, migration and blood vessel development (FIG. 2G). Additionally, a number of genes encoding transmembrane receptors, soluble factors, transcription factors and enzymes were differentially expressed; the scavenger receptor MARCO, TREM1, FCG2RB and IL21RG were up-regulated in En-TAM as compared to En-RM (FIG. 2H).

To better understand TAMs in different cancer types, we compared the gene expression profiles of Br-TAM and En-TAM. PCA and hierarchical clustering revealed two distinct groups (FIG. 3A) with very few DEGs commonly up- and down-regulated (18 genes up and 35 down), indicating that breast and endometrial cancers activate cancer tissue-specific transcriptional profiles in TAMs. Resident macrophages from endometrial and breast tissue also exhibited a distinct transcriptional profile confirming the diversity of tissue macrophage phenotypes in homeostatic states (FIG. 3B).

Macrophages exhibit distinct phenotypes in response to environmental stimuli and have been classified into two alternative polarization states, referred to as ‘M1’ and ‘M2’ with the latter being immune suppressive and pro-tumoral (10). To determine whether these polarization states exist within human En- and Br-TAM, we performed gene set enrichment analysis (GSEA) using the M1/M2 signature as proposed by Martinez et al. Neither Br- nor En-TAM showed a preferential enrichment for M2-associated genes supporting the idea that TAM phenotypes are much more complex and cannot be simply categorized into binary states (FIG. 3C).

### TAM Gene Signature is Enriched in Aggressive Breast Cancer Tumors

Increased density of TAMs has been associated with poor clinical outcomes in many human cancers (6). Importantly, studies using transcriptomic datasets have identified immune cell-specific gene sets to deconvolute the tumor microenvironment and its role in cancer progression (25). Taking advantage of a previously defined and validated compendium of immune cells (15,17), we sought to identify a TAM-specific immune signature. We focused on Br-TAM as breast cancer has greater number of in depth studies published. We selected upregulated genes in Br-TAM compared to Br-RM (Log₂FC>3, FDR<=0.05) that were also highly co-expressed in the METABRIC cohort (n=1350)(14), while filtering out genes belonging to other immune cell types (17) or those expressed by cancer cells. As a result, we identified a 37-gene TAM signature (Table 1). We performed whole tumor RNA-seq on an independent cohort of 47 breast cancer patients and evaluated the expression of our TAM signature (cohort 3). A previous study of breast cancer, defined a 112-gene CSF1 response signature associated with higher tumor grade, decreased expression of estrogen and progesterone receptor and higher mutation rate (23). Using this CSF1-response signature, we stratified our dataset (cohort) 3 into 3 groups (CSF1-High, -Mid and -Low). The TAM signature was significantly higher in the CSF1-High compared to -Mid and -Low groups suggesting that TAMs are associated with more aggressive tumors (FIG. 4A).

The samples were assigned to breast cancer molecular subtypes based on the PAM50 classification (24) with the TAM signature showing significantly higher expression in HER2 compared to Luminal A or B samples (p=0.02) (FIG. 4B).

We investigated whether the identified TAM signature was associated with clinical outcome in the METABRIC cohort. We observed a higher expression of the TAM signature in Basal, claudin-low, HER2 and Luminal B compared to Luminal A tumors, again showing an association of the TAM signature with more aggressive tumors (FIG. 4C). Consistent with these data, high expression of the TAM signature was significantly associated with shorter disease-specific survival (HR=1.3, p=0.006) (FIG. 4D). A previously reported macrophage immune signature (15,17), consisting mainly of lineage markers, showed a similar trend of high expression in aggressive tumors, but was not significantly associated with disease-specific survival (HR=1.17, p=0.1) (FIG. 4E). Taken together, these results suggest a positive association of unique populations of TAMs with poor clinical outcomes and more aggressive breast cancers.

### 3. Discussion

Despite the strong evidence for pro-tumoral roles of TAMs in mouse models of cancer (6) little is known about them in humans. Thus, we profiled TAMs in breast and endometrial cancers. Surprisingly, in contrast to monocytes, TAM transcriptomes from endometrial and breast cancers are distinct from each other, from their respective resident macrophages and their progenitor monocytes. These data suggest the existence of cancer specific niches that influence the TAM transcriptional profile according to tumor location and subtype. High expression of macrophage gene signatures has been associated with high tumor grade and poor clinical outcomes (25). In our study, we identified a 37-gene TAM signature that is highly expressed in the most aggressive breast cancer subtypes and enriched in a CSF1-high group that has been previously associated with higher tumor grade, decreased expression of estrogen and progesterone receptor, and higher mutation rate (23). Compared to a pan-macrophage immune signature (15,17), the TAM signature was associated with shorter disease-specific survival in the METABRIC cohort. These results, along with recent evidence of the role of TAMs in chemo- and immunotherapy resistance (26), highlight the need to study TAMs in human cancers and to identify markers for TAM-specific targeting.

### 4. References

1. http://www.breastcancer.org.
2. Breast cancer incidence statistics. http://www.cancerresearchuk.org/cancer-info/cancerstats/types/breast/incidence/uk-breast-cancer-incidence-statistics
3. Lauby-Secretan B, Loomis D, Straif K. Breast-Cancer Screening—Viewpoint of the IARC Working Group. N Engl J Med 2015; 373:1479
4. Noy R, Pollard J W. Tumor-associated macrophages: from mechanisms to therapy. Immunity 2014; 41:49-61
5. Cassetta L, Kitamura T. Targeting Tumor-Associated Macrophages as a Potential Strategy to Enhance the Response to Immune Checkpoint Inhibitors. Front Cell Dev Biol 2018; 6:38
6. Cassetta L, Pollard J W. Targeting macrophages: therapeutic approaches in cancer. Nat Rev Drug Discov 2018
7. Jiang X Q, Zhang L, Liu H A, Yuan N, Hou P Q, Zhang R Q, et al. Expansion of CD14(+)CD16(+) monocytes is related to acute leukemia. Int J Clin Exp Med 2015; 8:12297-306
8. Feng A L, Zhu J K, Sun J T, Yang M X, Neckenig M R, Wang X W, et al. CD16+ monocytes in breast cancer patients: expanded by monocyte chemoattractant protein-1 and may be useful for early diagnosis. Clin Exp Immunol 2011; 164:57-65
9. Qian B Z, Li J, Zhang H, Kitamura T, Zhang J, Campion L R, et al. CCL2 recruits inflammatory monocytes to facilitate breast-tumour metastasis. Nature 2011; 475:222-5
10. Martinez F O, Gordon S, Locati M, Mantovani A. Transcriptional profiling of the human monocyte-to-macrophage differentiation and polarization: new molecules and patterns of gene expression. J Immunol 2006; 177:7303-11
11. Cassetta L, Noy R, Swierczak A, Sugano G, Smith H, Wiechmann L, et al. Isolation of Mouse and Human Tumor-Associated Macrophages. Adv Exp Med Biol 2016; 899:211-29
12. Karnoub A E, Dash A B, Vo A P, Sullivan A, Brooks M W, Bell G W, et al. Mesenchymal stem cells within tumour stroma promote breast cancer metastasis. Nature 2007; 449:557-63
13. Finak G, Bertos N, Pepin F, Sadekova S, Souleimanova M, Zhao H, et al. Stromal gene expression predicts clinical outcome in breast cancer. Nat Med 2008; 14:518-27
14. Curtis C, Shah S P, Chin S F, Turashvili G, Rueda O M, Dunning M J, et al. The genomic and transcriptomic architecture of 2,000 breast tumours reveals novel subgroups. Nature 2012; 486:346-52
15. Bindea G, Mlecnik B, Tosolini M, Kirilovsky A, Waldner M, Obenauf A C, et al. Spatiotemporal dynamics of intratumoral immune cells reveal the immune landscape in human cancer. Immunity 2013; 39:782-95
16. Charoentong P, Finotello F, Angelova M, Mayer C, Efremova M, Rieder D, et al. Pan-cancer Immunogenomic Analyses Reveal Genotype-Immunophenotype Relationships and Predictors of Response to Checkpoint Blockade. Cell Rep 2017; 18:248-62
17. Tamborero D, Rubio-Perez C, Muinos F, Sabarinathan R, Piulats J M, Muntasell A, et al. A Pan-cancer Landscape of Interactions between Solid Tumors and Infiltrating Immune Cell Populations. Clin Cancer Res 2018
18. M K. Caret: Classification and Regression Training. Astrophysics Source Code 50 library; 2015.
19. Dominic P, Ajit N, Tom F, Andrew S. Continuous Biomarker Assessment by Exhaustive Survival Analysis. BioRxiv 2018
20. Bolger A M, Lohse M, Usadel B. Trimmomatic: a flexible trimmer for Illumina sequence data. Bioinformatics 2014; 30:2114-20
21. Kim S, Jeong K, Bhutani K, Lee J, Patel A, Scott E, et al. Virmid: accurate detection of somatic mutations with sample impurity inference. Genome Biol 2013; 14:R90
22. Roberts A, Trapnell C, Donaghey J, Rinn J L, Pachter L. Improving RNA-Seq expression estimates by correcting for fragment bias. Genome Biol 2011; 12:R22
23. Beck A H, Espinosa I, Edris B, Li R, Montgomery K, Zhu S, et al. The macrophage colony-stimulating factor 1 response signature in breast carcinoma. Clin Cancer Res 2009; 15:778-87
24. Parker J S, Mullins M, Cheang M C, Leung S, Voduc D, Vickery T, et al. Supervised risk predictor of breast cancer based on intrinsic subtypes. J Clin Oncol 2009; 27:1160-7
25. Gentles A J, Newman A M, Liu C L, Bratman S V, Feng W, Kim D, et al. The prognostic landscape of genes and infiltrating immune cells across human cancers. Nat Med 2015; 21:938-45
26. Neubert N J, Schmittnaegel M, Bordry N, Nassiri S, Wald N, Martignier C, et al. T cell-induced CSF1 promotes melanoma resistance to PD1 blockade. Sci Transl Med 2018; 10

1. A method of diagnosing and/or prognosing breast cancer, predicting efficacy of treatment for breast cancer, assessing outcome of treatment for breast cancer and/or assessing recurrence of breast cancer, the method comprising:
a) analysing a biological sample obtained from a subject to determine the presence of target molecules representative of expression of at least two biomarkers selected from the group listed in Table 1, wherein the biological sample is a breast tissue sample or derivative thereof; and
b) comparing the expression levels of the biomarkers determined in (a) with one or more reference values, wherein whether there is a difference in the expression of the biomarkers in the sample from the subject compared to the one or more reference values is indicative of a clinical indication.

2. A method according to claim 1 wherein the at least two biomarkers comprise a biomarker for SIGLEC1.

3. A method according to claim 1 or claim 2 wherein the at least two biomarkers comprise at least two biomarkers from Table 2.

4. A method according to any of claims 1 to 3 wherein said clinical indication comprises one or more of the presence or absence of breast cancer in the breast tissue sample from the subject, the receptor status of breast cancer in the tissue sample, for example oestrogen (ER), HER2 and/or progesterone receptor status, tumor grade of breast cancer in the tissue sample, likelihood of metastasis from breast cancer in the tissue sample, likely outcome of treatment of the breast cancer in the subject, likelihood of recurrence of the breast cancer following treatment, an indication of whether the prognosis for the breast cancer and subject is good or poor and/or predicted survival (life expectancy) of the subject.

5. A method according to any preceding claim wherein one or more of the reference values are associated with a particular clinical indication such that a defined difference in the expression of the biomarkers in the sample from the subject compared to the one or more reference values is indicative of a particular clinical indication.

6. A method according to claim 5 wherein the reference values may be representative of the expression of the same biomarkers in resident macrophages from breast tissue of subjects not having breast cancer, and a diagnosis that the subject has breast cancer will be indicated when there is differential expression of the biomarkers compared to the corresponding biomarker reference values, and/or a diagnosis that the subject does not have breast cancer will be indicated when there is substantially no differential expression.

7. A method according to any of claims 1 to 4 wherein the reference values are in the form of gene expression signatures corresponding to the biomarker expression levels in macrophages from breast tissue having a known particular clinical indication, and a difference in the expression levels of the biomarkers in the biological sample will be assessed by determining whether said expression levels of the biomarkers of the biological sample correlate with one of the gene expression signatures, thereby stratifying the biological sample breast tissue as being of the same clinical indication as that with which the gene expression signature is correlated.

8. A method according to claim 7 wherein the expression levels of the biomarkers determined in (a) may be compared with one or more gene expression signatures representing gene expression levels of the same biomarkers in macrophages from breast cancer tissue having a good outcome and one or more gene expression signatures representing gene expression levels of the same biomarkers in macrophages from breast cancer tissue having a poor outcome,
and wherein the biological sample breast tissue will be indicated as being associated with a poor outcome if it stratifies with the poor outcome gene expression signature, and indicated as being associated with a good outcome if it stratifies with the good outcome gene expression signature.

9. A method of treating breast cancer in a subject, comprising:
a) analysing a biological sample obtained from a subject to determine the presence of target molecules representative of expression of at least two biomarkers selected from the group listed in Table 1;
b) comparing the expression levels of the biomarkers determined in (a) with one or more reference values, and
providing the subject with a particular treatment for breast cancer according to whether there is a difference in the expression of the biomarkers in the sample from the subject compared to the one or more reference values.

10. A method according to claim 9 wherein the at least two biomarkers comprise a biomarker for SIGLEC1.

11. A method according to claim 9 or claim 10 wherein the at least two biomarkers comprise at least two biomarkers from Table 2.

12. A method according to any of claims 9 to 11 wherein said clinical indication comprises one or more of the presence or absence of breast cancer in the breast tissue sample from the subject, the receptor status of breast cancer in the tissue sample, for example oestrogen (ER), HER2 and/or progesterone receptor status, tumor grade of breast cancer in the tissue sample, likelihood of metastasis from breast cancer in the tissue sample, likely outcome of treatment of the breast cancer in the subject, likelihood of recurrence of the breast cancer following treatment, an indication of whether the prognosis for the breast cancer and subject is good or poor and/or predicted survival (life expectancy) of the subject.

13. A method according to any of claims 9 to 12 wherein one or more of the reference values are associated with a particular clinical indication such that a defined difference in the expression of the biomarkers in the sample from the subject compared to the one or more reference values is indicative of a particular clinical indication.

14. A method according to claim 13 wherein the reference values may be representative of the expression of the same biomarkers in resident macrophages from breast tissue of subjects not having breast cancer, and a diagnosis that the subject has breast cancer will be indicated when there is differential expression of the biomarkers compared to the corresponding biomarker reference values, and/or a diagnosis that the subject does not have breast cancer will be indicated when there is substantially no differential expression.

15. A method according to any of claims 9 to 12 wherein the reference values are in the form of gene expression signatures corresponding to the biomarker expression levels in macrophages from breast tissue having a known particular clinical indication, and a difference in the expression levels of the biomarkers in the biological sample will be assessed by determining whether said expression levels of the biomarkers of the biological sample correlate with one of the gene expression signatures, thereby stratifying the biological sample breast tissue as being of the same clinical indication as that with which the gene expression signature is correlated.

16. A method according to claim 15 wherein the expression levels of the biomarkers determined in (a) may be compared with one or more gene expression signatures representing gene expression levels of the same biomarkers in macrophages from breast cancer tissue having a good outcome and one or more gene expression signatures representing gene expression levels of the same biomarkers in macrophages from breast cancer tissue having a poor outcome,
and wherein the biological sample breast tissue will be indicated as being associated with a poor outcome if it stratifies with the poor outcome gene expression signature, and indicated as being associated with a good outcome if it stratifies with the good outcome gene expression signature.

17. A kit comprising binding partners capable of binding to target molecules representative of expression of at least two biomarkers selected from the group listed in Table 1.

18. A kit according to claim 17 comprising binding partners capable of binding to target molecules representative of expression of at least two biomarkers selected from the group listed in Table 2.

19. A kit according to claim 17 or claim 18 further comprising indicators capable of indicating when said binding occurs.

20. A kit according to any of claims 17 to 19 wherein the at least two biomarkers comprise a biomarker for SIGLEC1.

21. An assay device comprising:
a) a loading area for receipt of a biological sample;
b) binding partners specific for target molecules representative of expression of at least two biomarkers selected from the group listed in Table 1; and
c) detection means to detect the levels of said target molecules present in the sample.

22. An assay device according to claim 21 wherein the at least two biomarkers comprise a biomarker for SIGLEC1.

23. An assay device according to claim 21 or claim 22 wherein the at least two biomarkers comprise at least two biomarkers from Table 2.


SEQUENCE LISTING (37 sequences)

SEQ ID NO: 1 - DNA, 50 bp, Artificial Sequence
  Probe Sequence (Illumina Human HT 12 V4 probes) - ILMN_1737918
accaacaaggggctcttccaggtggtgtcagggggcatggtgcttcagct

SEQ ID NO: 2 - DNA, 50 bp, Artificial Sequence
  Probe Sequence (Illumina Human HT 12 V4 probes) - ILMN_1796409
caggccaccgacaagaactcactactgggcatggagggtgccaacagcat

SEQ ID NO: 3 - DNA, 50 bp, Artificial Sequence
  Probe Sequence (Illumina Human HT 12 V4 probes) - ILMN_181786
gccagatatgcctgtttccttttcccagcaccatgcctgtggaggggaca

SEQ ID NO: 4 - DNA, 50 bp, Artificial Sequence
  Probe Sequence (Illumina Human HT 12 V4 probes) - ILMN_25185
caggattccatggaccacctggacaagcaaacccaaactccgaagacttg

SEQ ID NO: 5 - DNA, 50 bp, Artificial Sequence
  Probe Sequence (Illumina Human HT 12 V4 probes) - ILMN_24462
gcaggaacaacagatgcaggaacaggctgcacagctcagcacaacattcc

SEQ ID NO: 6 - DNA, 50 bp, Artificial Sequence
  Probe Sequence (Illumina Human HT 12 V4 probes) - ILMN_8098
ggtggcacaggaacccccgccccaacttttggattgtaataaaacaattg

SEQ ID NO: 7 - DNA, 50 bp, Artificial Sequence
  Probe Sequence (Illumina Human HT 12 V4 probes) - ILMN_20705
ccttcttcctcccaggaaaggggacgttcagctgagccgagtgtgtatac

SEQ ID NO: 8 - DNA, 50 bp, Artificial Sequence
  Probe Sequence (Illumina Human HT 12 V4 probes) - ILMN_17792
gtctccccacctctaccagcatctgctgagctatgagccaaaccagggat

SEQ ID NO: 9 - DNA, 50 bp, Artificial Sequence
  Probe Sequence (Illumina Human HT 12 V4 probes) - ILMN_12611
acaagctgtccccgctgcacgagcgcatctactgtgcactctctggttca

SEQ ID NO: 10 - DNA, 50 bp, Artificial Sequence
  Probe Sequence (Illumina Human HT 12 V4 probes) - ILMN_172048
ccatgggccttttcacaggggacacaggcttcttaaaacaacccggcttc

SEQ ID NO: 11 - DNA, 50 bp, Artificial Sequence
  Probe Sequence (Illumina Human HT 12 V4 probes) - ILMN_28413
gttctccagaggaaggtggaagaaaccatgggcaggagtaggaattgagt

SEQ ID NO: 12 - DNA, 50 bp, Artificial Sequence
  Probe Sequence (Illumina Human HT 12 V4 probes) - ILMN_27857
ggccaaacttggagcaggtgtccatcccagccctgtgtagttagagcagg

SEQ ID NO: 13 - DNA, 50 bp, Artificial Sequence
  Probe Sequence (Illumina Human HT 12 V4 probes) - ILMN_138034
aggtgtcatttccattatttatattagtttagccaaaggataagtgtcct

SEQ ID NO: 14 - DNA, 50 bp, Artificial Sequence
  Probe Sequence (Illumina Human HT 12 V4 probes) - ILMN_175406
caggctactttccgtccatgtacctgcaaaagtcggggcaagacgtgtcc

SEQ ID NO: 15 - DNA, 50 bp, Artificial Sequence
  Probe Sequence (Illumina Human HT 12 V4 probes) - ILMN_12514
ccaacaaagatgaagttccctatctacggaaaggcatgactgggaggccc

SEQ ID NO: 16 - DNA, 50 bp, Artificial Sequence
  Probe Sequence (Illumina Human HT 12 V4 probes) - ILMN_2315
ccccagagataaaggctgccattttgggggtctgtacttatggcctgaaa

SEQ ID NO: 17 - DNA, 50 bp, Artificial Sequence
  Probe Sequence (Illumina Human HT 12 V4 probes) - ILMN_138267
ggagacttgaggagggcttgaggttggtgaggttaggtgcgtgtttcctg

SEQ ID NO: 18 - DNA, 50 bp, Artificial Sequence
  Probe Sequence (Illumina Human HT 12 V4 probes) - ILMN_38361
ctcctggtcctcttctgcctccatcgccagaatcagataaagcaggggcc

SEQ ID NO: 19 - DNA, 50 bp, Artificial Sequence
  Probe Sequence (Illumina Human HT 12 V4 probes) - ILMN_11875
gaatgctggtgagatgcttcatgggactgggggtctcctgctcagtctgg

SEQ ID NO: 20 - DNA, 50 bp, Artificial Sequence
  Probe Sequence (Illumina Human HT 12 V4 probes) - ILMN_181889
tgggtgctcgcccacttgtcccactatctgggtgcatgatcttgagcaag

SEQ ID NO: 21 - DNA, 50 bp, Artificial Sequence
  Probe Sequence (Illumina Human HT 12 V4 probes) - ILMN_34388
gagaccacgcagctcattgatcctgatgcagccacatgtgagacctcaac

SEQ ID NO: 22 - DNA, 50 bp, Artificial Sequence
  Probe Sequence (Illumina Human HT 12 V4 probes) - ILMN_6136
ctgcaggtctcccatgaaggccaccccatggtctgatgggcatgaagcat

SEQ ID NO: 23 - DNA, 50 bp, Artificial Sequence
  Probe Sequence (Illumina Human HT 12 V4 probes) - ILMN_20706
ttgccaaagggctgcgagaggtgattgagaagaaccggcctgctgatgtc

SEQ ID NO: 24 - DNA, 50 bp, Artificial Sequence
  Probe Sequence (Illumina Human HT 12 V4 probes) - ILMN_21820
gagccggccctgcatggaggaaaatgaattgtacagcagcgcagtcacag

SEQ ID NO: 25 - DNA, 50 bp, Artificial Sequence
  Probe Sequence (Illumina Human HT 12 V4 probes) - ILMN_1865
gccatcccccatcttccctagacacagcagacatctgagaaagcttcagc

SEQ ID NO: 26 - DNA, 50 bp, Artificial Sequence
  Probe Sequence (Illumina Human HT 12 V4 probes) - NM_152854.2
ctggaagggtacacagaaaacccacagctcgaagagtggtgacgtctggg

SEQ ID NO: 27 - DNA, 50 bp, Artificial Sequence
  Probe Sequence (Illumina Human HT 12 V4 probes) - ILMN_1999
cctctgcaccatggctctctgcaaccagttctctgcatcacttgctgctg

SEQ ID NO: 28 - DNA, 50 bp, Artificial Sequence
  Probe Sequence (Illumina Human HT 12 V4 probes) - ILMN_15634
gtcattgttctccctcctacctgtctgtagtgttgtggggtcctcccatg

SEQ ID NO: 29 - DNA, 50 bp, Artificial Sequence
  Probe Sequence (Illumina Human HT 12 V4 probes) - ILMN_11290
ccagctcagtcaagccgccacatgcccacaacctcaccagagggagaatt

SEQ ID NO: 30 - DNA, 50 bp, Artificial Sequence
  Probe Sequence (Illumina Human HT 12 V4 probes) - ILMN_28131
tgccccacaccctcccttaccctcctccagaccattcaggacacagggaa

SEQ ID NO: 31 - DNA, 50 bp, Artificial Sequence
  Probe Sequence (Illumina Human HT 12 V4 probes) - ILMN_20639
tcaagatccttaacaagaagggacagcaaggctggtggcgaggggagatc

SEQ ID NO: 32 - DNA, 50 bp, Artificial Sequence
  Probe Sequence (Illumina Human HT 12 V4 probes) - ILMN_3324
cagcgtgcatgtgttcaagccttagattggcgatgtcgtattttcctcac

SEQ ID NO: 33 - DNA, 50 bp, Artificial Sequence
  Probe Sequence (Illumina Human HT 12 V4 probes) - ILMN_13097
agagagcaaactaccacaaccaatggttgagcccctgtcaagtgccagtc

SEQ ID NO: 34 - DNA, 50 bp, Artificial Sequence
  Probe Sequence (Illumina Human HT 12 V4 probes) - ILMN_22632
tgctgagaacaaacccacctgagcaccccagacaccttcctcaacccagg

SEQ ID NO: 35 - DNA, 50 bp, Artificial Sequence
  Probe Sequence (Illumina Human HT 12 V4 probes) - ILMN_18144
ggcccttctagtatctctgccgggggcttctggtactcctctctaaatac

SEQ ID NO: 36 - DNA, 50 bp, Artificial Sequence
  Probe Sequence (Illumina Human HT 12 V4 probes) - ILMN_33785
gactcagagattctcttttgtccacagacagtcatctcaggagcagaaag

SEQ ID NO: 37 - DNA, 50 bp, Artificial Sequence
  Probe Sequence (Illumina Human HT 12 V4 probes) - ILMN_18780
atggtgggcatccagggctctgacagcgtcttctccggcttcctgctctt